(19) United States
(12) Patent Application Publication
(10) Pub. No.: US 2021/0290512 A1
(43) Pub. Date: Sep. 23, 2021

(54) METHOD FOR DYEING KERATINOUS MATERIAL, COMPRISING THE USE OF AN ORGANOSILICON COMPOUND, A SURFACTANT AND A PIGMENT

(71) Applicant: Henkel AG & Co. KGaA, Duesseldorf (DE)

(72) Inventors: TORSTEN LECHNER, Langenfeld (DE); JUERGEN SCHOEPGENS, Schwalmtal (DE); MARC NOWOTTNY, Moenchengladbach (DE); GABRIELE WESER, Neuss (DE); ULRIKE SCHUMACHER, Duesseldorf (DE); CLAUDIA KOLONKO, Remscheid (DE); CAROLINE KRIENER, Duesseldorf (DE); CARSTEN MATHIASZYK, Essen (DE)

(73) Assignee: Henkel AG & Co. KGaA, Duesseldorf (DE)

(21) Appl. No.: 17/267,956

(22) PCT Filed: May 22, 2019
(86) PCT No.: PCT/EP2019/063202

§ 371 Date: Jun. 2, 2021

(30) Foreign Application Priority Data
Aug. 16, 2018 (DE) 10 2018 213 814.1

(51) Int. Cl. A61K 8/58; A61K 8/41; A61K 8/49; A61K 8/19; A61K 8/29; A61K 8/81; A61Q 5/06; A45D 19/00

(52) U.S. Cl. CPC A61K 8/585; A61K 8/416; A61K 8/4946; A61K 8/19; A61K 2800/884; A61K 8/8158; A61Q 5/065; A45D 19/0066; A61K 2800/43; A61K 8/29

(57) ABSTRACT

The subject of the present invention is a process for dyeing keratinous material, in particular human hair, comprising the following steps:
Application of an agent (a) to the keratinous material, the agent (a) containing at least one organic silicon compound and at least one surfactant, and
Application of an agent (b) to the keratinous material, the agent (b) containing at least one pigment.

A second object of the present invention is a multi-component packaging unit containing means (a) and (b) in three separately assembled containers.

The subject of the present application is a process for dyeing keratinous material, especially human hair, which comprises the application of at least two different agents (a) and (b). The agent (a) contains at least one organic silicon compound and at least one surfactant. The agent (b) contains at least one pigment.

The second subject-matter of this application is a multi-component packaging unit (kit-of-parts) for coloring keratinous material, in particular human hair, which comprises the agents (a) and (b) separately packaged in two different containers.

Changing the shape and color of keratinous material, especially human hair, is an important area of modern cosmetics. To change the hair color, the expert knows various coloring systems depending on the coloring requirements. Oxidation dyes are usually used for permanent, intensive dyeing's with good fastness properties and good grey coverage. Such colorants contain oxidation dye precursors, so-called developer components and coupler components, which, under the influence of oxidizing agents such as hydrogen peroxide, form the actual dyes among themselves. Oxidation dyes are characterized by very long-lasting dyeing results.

When direct dyes are used, ready-made dyes diffuse from the colorant into the hair fiber. Compared to oxidative hair dyeing, the dyeing's obtained with direct dyes have a shorter shelf life and quicker wash ability. Dyeing with direct dyes usually remain on the hair for a period of between 5 and 20 washes.

The use of color pigments is known for short-term color changes on the hair and/or skin. Color pigments are generally understood to be insoluble, coloring substances. These are present undissolved in the dye formulation in the form of small particles and are only deposited from the outside on the hair fibers and/or the skin surface. Therefore, they can usually be removed again without residue by a few washes with detergents containing surfactants. Various products of this type are available on the market under the name hair mascara.

If the user wants particularly long-lasting dyeing's, the use of oxidative dyes has so far been his only option. However, despite numerous optimization attempts, an unpleasant ammonia or amine odor cannot be completely avoided in oxidative hair dyeing. The hair damage still associated with the use of oxidative dyes also has a negative effect on the user's hair. A continuing challenge is therefore the search for alternative, high-performance dyeing processes.

EP 2168633 B1 deals with the task of producing long-lasting hair colorations using pigments. It teaches that by using a combination of pigment, organic silicon compound, hydrophobic polymer, and a solvent, it is possible to create colorations on hair that are particularly resistant to shampooing. For example, 3-aminopropyl-triethoxysilane was used as organic silicon compound.

In WO 2018/115059 A1 a dyeing process is described, which runs in several steps. One step involves the application of a direct dye, and in a further step an organosilane is applied to the hair. This process is also used to achieve dyeing's with good wash fastness properties. The silanes used in this writing are for example 3-aminopropyltriethxoysilane and methyltrimethoxysilane.

In the dyeing processes of EP 2168633 B1 and WO 2018/115059 A1, organosilicon compounds from the group of silanes are used, the molecular structure of these silanes comprising at least one hydroxy group and/or hydrolysable group. Due to the presence of hydroxy groups or hydrolysable groups, silanes are reactive substances that hydrolyze or oligomerize or polymerize in the presence of water. When applied to the keratin material, the oligomerization or polymerization of the silanes initiated by the presence of water ultimately leads to the formation of a film which fixes the coloring compounds and thus produces very long-lasting colorations.

Closer examination of the dyeing processes disclosed in EP 2168633 B1 and WO 2018/115059 A1 has shown that the dyeing results obtained in these dyeing processes still need improvement. Methods are still being sought to further improve the color intensity of dyeing's obtained by using pigments. In addition, it is a previously unsolved problem to achieve a brightening effect with the use of pigments.

The purpose of the present intention was to provide a dyeing system with fastness properties comparable to those of oxidative dyeing. Wash fastness properties should be outstanding, but the use of oxidation dye precursors normally used for this purpose should be avoided. A technology was sought that would make it possible to fix the coloring compounds (such as pigments) known from the state of the art in an extremely durable way to the hair. This should result in particularly even colorations with high color intensity. In addition, a color result should be achieved with the application of the procedure, which can also be lighter than the original hair color. In other words, it should be possible to lighten the hair with the use of pigments, giving it the appearance of natural bleaching.

Surprisingly, it has now turned out that the above-mentioned task can be excellently solved if keratinous materials, especially hair, are dyed using a procedure in which at least two agents (a) and (b) are applied to the keratinous materials (hair). The agent (a) contains at least one organic silicon compound and in addition at least one surfactant. The agent (b) contains at least one pigment. When using both agents (a) and (b) in one dyeing process, dyeing's with particularly high color intensity and great uniformity were obtained. The leveling ability of these stains was excellent.

When dyeing dark hair strands with the method according to the invention, a lighter coloration of the hair strands could also be achieved if light pigments were used in agent (b). Due to the great uniformity of the color result, the blond colors obtained in this way looked very natural.

A first subject matter of the present invention is therefore a process for dyeing keratinous material, in particular human hair, comprising the following steps:
Application of an agent (a) to the keratinous material, the agent (a) containing at least one organic silicon compound and at least one surfactant, and
Application of an agent (b) to the keratinous material, the agent (b) containing at least one pigment.

### Keratinic Material

Keratinous material includes hair, skin, nails (such as fingernails and/or toenails). Wool, furs, and feathers also fall under the definition of keratinous material.

Preferably, keratinous material is understood to be human hair, human skin, and human nails, especially fingernails and toenails. Keratinous material is understood to be human hair.

### Agent (a) and (b)

In the procedure according to the invention, agents (a) and (b) are applied to the keratinous material, in particular human hair. The two means (a) and (b) are different.

In other words, a first subject of the present invention is a process for dyeing keratinous material, in particular human hair, comprising the following steps:
Application of an agent (a) to the keratinous material, the agent (a) containing at least one organic silicon compound and at least one surfactant, and
Application of an agent (b) to the keratinous material, the agent (b) containing at least one pigment,
   where the two means (a) and (b) are different.

### Agent (a)

As the first ingredient essential to the invention, the agent contains (a) at least one organic silicon compound.

In particular, the composition contains (a) at least one organic silicon compound selected from silanes having one, two or three silicon atoms, the organic silicon compound comprising one or more hydroxyl groups and/or hydrolysable groups per molecule.

These organic silicon compounds or organic silanes contained in average (a) are reactive compounds.

Composition (a) contains the organic silicon compound(s), in particular the organic silane(s), in a cosmetic carrier which may be hydrated, low in water or anhydrous. In addition, the cosmetic carrier can be liquid, gel-like, creamy, powdery, or even solid (e.g., in the form of a tablet or pellet). Preferably, the cosmetic carrier of the product (a) is an aqueous or aqueous-alcoholic carrier. To hair coloration, such carriers are, for example, creams, emulsions, gels, or surfactant-containing foaming solutions, such as shampoos, foam aerosols, foam formulations or other preparations suitable for application to the hair.

The cosmetic carrier preferably contains water, which means that the carrier contains at least 2% by weight of water based on its weight. Preferably, the water content is above 5 wt. %, further preferably above 10 wt. % still further preferably above 15 wt. %. The cosmetic carrier can also be aqueous alcoholic. [0206] Aqueous/alcoholic solutions in the context of the invention are aqueous solutions containing 2 to 70% by weight of a C₁-C₄ alcohol, more particularly ethanol or isopropanol. The agents according to the invention may additionally contain other organic solvents, such as methoxy butanol, benzyl alcohol, ethyl diglycol or 1,2-propylene glycol. Preferred are all water-soluble organic solvents.

The term “coloring agent” is used in the context of this invention for a coloring of the keratin material, in particular the hair, caused using pigments. In this coloration, the pigments are deposited in a particularly homogeneous, even, and smooth film on the surface of the keratin material. The film is formed in situ by oligomerization or polymerization of the organic silicon compound(s) and by the interaction of organic silicon compound with the pigment(s).

### Organic Silicon Compounds

As the first ingredient essential to the invention, the agent (a) contains at least one organic silicon compound. Preferred organic silicon compounds are selected from silanes with one, two or three silicon atoms, where the organic silicon compound comprises one or more hydroxyl groups and/or hydrolysable groups per molecule.

Organic silicon compounds, alternatively called organosilicon compounds, are compounds which either have a direct silicon-carbon bond (Si—C) or in which the carbon is bonded to the silicon atom via an oxygen, nitrogen, or sulfur atom. The organic silicon compounds according to the invention are compounds containing one to three silicon atoms. Organic silicon compounds preferably contain one or two silicon atoms.

According to IUPACrules, the term silane stands for a group of chemical compounds based on a silicon skeleton and hydrogen. In organic silanes, the hydrogen atoms are completely or partially replaced by organic groups such as (substituted) alkyl groups and/or alkoxy groups. In organic silanes, some of the hydrogen atoms may also be replaced by hydroxy groups.

Composition (a) contains at least one organic silicon compound selected from silanes having one, two or three silicon atoms, wherein the organic silicon compound comprises one or more hydroxyl groups or hydrolysable groups per molecule.

In a particularly preferred embodiment, a method according to the invention is characterized by the application of an agent (a) to the keratinous material, said agent (a) comprising at least one organic silicon compound selected from silanes having one, two or three silicon atoms, said organic silicon compound further comprising one or more basic chemical functions and one or more hydroxyl groups or hydrolysable groups per molecule.

This basic group can be, for example, an amino group, an alkylamino group or a dialkylamino group, which is preferably connected to a silicon atom via a linker. The basic group is preferably an amino group, a C₁-C₆ alkylamino group or a di(C₁-C₆)alkylamino group.

The hydrolysable group(s) is (are) preferably a C₁-C₆ alkoxy group, especially an ethoxy group or a methoxy group. It is preferred when the hydrolysable group is directly bonded to the silicon atom. For example, if the hydrolysable group is an ethoxy group, the organic silicon compound preferably contains a structural unit R′R″R′″Si—O—CH2-CH3. The residues R′, R″ and R′″ represent the three remaining free valences of the silicon atom.

A particularly preferred method according to the invention is characterized in that the composition comprises (a) at least one organic silicon compound selected from silanes having one, two or three silicon atoms, the organic silicon compound preferably comprising one or more basic chemical functions and one or more hydroxyl groups or hydrolysable groups per molecule.

Particularly good results could be obtained if the agent according to the invention (a) contains at least one organic silicon compound of formula (I) and/or (II).

The compounds of formulae (I) and (II) are organic silicon compounds selected from silanes having one, two or three silicon atoms, the organic silicon compound comprising one or more hydroxyl groups and/or hydrolysable groups per molecule.

In another particularly preferred embodiment, a method according to the invention is characterized in that an agent (a) is applied to the keratinous material (or human hair), the agent (a) containing at least one organic silicon compound of formula (I) and/or (II).

R₁R₂N-L-Si(OR₃)ₐ(R₄)_{b}
where
   R₁, R₂ independently represent a hydrogen atom or a C₁-C₆ alkyl group,
   L is a linear or branched divalent C₁-C₂₀ alkylene group,
   R3 represents a hydrogen atom or a C₁-C alkyl group
   R4 represents a C1-C6 alkyl group
   a, stands for an integer from 1 to 3, and
   b stands for the integer 3−a,

(R₅O)_{c}(R₆)_{d}Si-(A)ₑ-[NR₇-(A′)]_{f}-[O-(A″)]_{g}-[NR₈-(A′″)]ₕ-Si(R_{6′})_{d′}(OR₅′)_{c′} (II),
where
   R5, R5′, R5″ independently represent a hydrogen atom or a C₁-C₆ alkyl group,
   R6, R6′ and R6″ independently represent a C₁-C₆ alkyl group,
   A, A′, A″, A′″ and A″″ independently of one another represent a linear or branched divalent C₁-C₂₀- Alkylene group
   R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III)

-(A″″)-Si(R₆″)_{d″}(OR₅″)_{c}″ (III),
c, stands for an integer from 1 to 3,
d stands for the integer 3−c,
c′ stands for an integer from 1 to 3,
d′ stands for the integer 3−c′,
c″ stands for an integer from 1 to 3,
d″ stands for the integer 3−c″,
e stands for 0 or 1,
f stands for 0 or 1,
g stands for 0 or 1,
h stands for 0 or 1,
provided that at least one of e, f, g, and h is different from 0.

The substituents R₁, R₂, R₃, R₄, R₅, R₅′, R₅″, R₆, R₆′, R₆″, R₇, R₈, L, A′, A″″ and A″″ in the compounds of formula (I) and (II) are explained below as examples:

Examples of a C₁-C₆ alkyl group are the groups methyl, ethyl, propyl, isopropyl, n-butyl, s-butyl, and t-butyl, n-pentyl and n-hexyl. Propyl, ethyl, and methyl are preferred alkyl radicals. Examples of a C₂-C₆ alkenyl group are vinyl, allyl, but-2-enyl, but-3-enyl and isobutenyl, preferred C₂-C₆ alkenyl radicals are vinyl and allyl. Preferred examples of a hydroxy C₁-C₆ alkyl group are a hydroxymethyl, a 2-hydroxyethyl, a 2-hydroxypropyl, a 3-hydroxypropyl, a 4-hydroxybutyl group, a 5-hydroxypentyl and a 6-hydroxyhexyl group; a 2-hydroxyethyl group is particularly preferred. Examples of an amino C₁-C₆ alkyl group are the aminomethyl group, the 2-aminoethyl group, the 3-aminopropyl group. The 2-aminoethyl group is particularly preferred. Examples of a linear divalent C₁-C₂₀ alkylene group include the methylene group (—CH₂),), the ethylene group (—CH₂—CH₂—), the propylene group (—CH₂—CH₂—CH₂—) and the butylene group (—CH₂—CH₂—CH₂—). The propylene group (—CH₂—CH₂—CH₂—) is particularly preferred. From a chain length of 3 C atoms, divalent alkylene groups can also be branched. Examples of branched divalent C₃-C₂₀ alkylene groups are (—CH₂—CH(CH₃)—) and (—CH₂—CH(CH₃)—CH₂—).

In the organic silicon compounds of the formula (I)

R₁R₂N-L-Si(OR₃)ₐ(R₄)_{b} (I),

the radicals R₁ and R₂ independently of one another represent a hydrogen atom or a C₁-C₆ alkyl group. In particular, the radicals R₁ and R₂ both represent a hydrogen atom.

In the middle part of the organic silicon compound is the structural unit or the linker -L- which stands for a linear or branched, divalent C₁-C₂₀ alkylene group.

Preferably -L- stands for a linear, divalent C₁-C₂₀ alkylene group. Further preferably -L- stands for a linear divalent C₁-C₆ alkylene group. Particularly preferred -L stands for a methylene group (CH₂—), an ethylene group (—CH₂—CH₂—), propylene group (—CH₂—CH₂—CH₂—) or butylene (—CH₂—CH₂—CH₂—CH₂—). L stands for a propylene group (—CH₂—CH₂—CH₂—)

The organic silicon compounds of formula (I)

R₁R₂N-L-Si(OR₃)ₐ(R₄)_{b} (I),

one end of each carries the silicon-containing group —Si(OR₃)ₐ(R₄)_{b}

In the terminal structural unit —Si(OR₃)ₐ(R₄)_{b}, R₃ is hydrogen or C₁-C₆ alkyl group, and R₄ is C₁-C₆ alkyl group. R3 and R₃ independently of each other represent a methyl group or an ethyl group.

Here a stands for an integer from 1 to 3, and b stands for the integer 3−a. If a stands for the number 3, then b is equal to 0. If a stands for the number 2, then b is equal to 1. If a stands for the number 1, then b is equal to 2.

Dyes with the best wash fastness values could be obtained if the pretreatment agent contains at least one organic silicon compound corresponding to formula (I): in which R₃, R₄ independently of one another represent a methyl group or an ethyl group.

Furthermore, dyeing's with the best wash fastness properties could be obtained if the agent according to the invention contains at least one organic silicon compound of formula (I) in which the radical a represents the number 3. In this case the rest b stands for the number 0.

In another preferred embodiment, an agent according to the invention is characterized in that it contains (a) at least one organic silicon compound of formula (I),
where
   R₃, R₄ independently of one another represent a methyl group or an ethyl group and
   a stands for the number 3 and
   b stands for the number 0.

In another preferred embodiment, a process according to the invention is characterized in that the agent (a) contains at least one organic silicon compound of formula (I),

R₁R₂N-L-Si(OR₃)ₐ(R₄)_{b} (I),
where
   R₁, R₂ both represent a hydrogen atom, and
   L represents a linear, divalent C₁-C₆-alkylene group, preferably a propylene group (—CH₂—CH₂—CH₂—) or an ethylene group (—CH₂—CH₂—),
   R₃ represents a hydrogen atom, an ethyl group, or a methyl group,
   R₄ represents a methyl group or an ethyl group,
   a stands for the number 3 and
   b stands for the number 0.

Organic silicon compounds of the formula (I) which are particularly suitable for solving the problem according to the invention are
(3-Aminopropyl)triethoxysilan

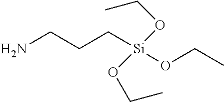

(3-Aminopropyl)trimethoxysilane

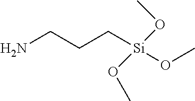

1-(3-Aminopropyl)silantriol

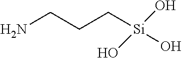

(2-Aminoethyl)triethoxysilan

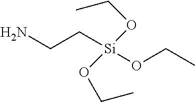

(2-Aminoethyl)trimethoxysilane

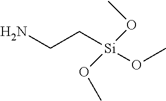

1-(2-Aminoethyl)silantriol

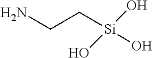

(3-Dimethylaminopropyl)triethoxysilan

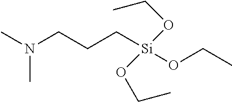 [SMILES: CCO[Si](CCCN(C)C)(OCC)OCC]

(3-Dimethylaminopropyl)trimethoxysilane

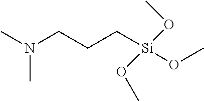

1-(3-Dimethylaminopropyl)silantriol

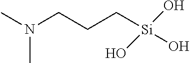

(2-Dimethylaminoethyl)triethoxysilan.

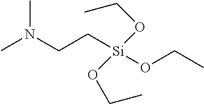

(2-Dimethylaminoethyl)trimethoxysilane and/or

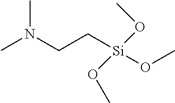

1-(2-Dimethylaminoethyl)silantriol

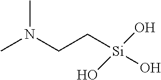

In another preferred embodiment, a process according to the invention is characterized in that the agent (a) contains at least one organic silicon compound selected from the group consisting of
(3-Aminopropyl)triethoxysilan
(3-Aminopropyl)trimethoxysilane
1-(3-Aminopropyl)silantriol
(2-Aminoethyl)triethoxysilan
(2-Aminoethyl)trimethoxysilane
1-(2-Aminoethyl)silantriol
(3-Dimethylaminopropyl)triethoxysilan
(3-Dimethylaminopropyl)trimethoxysilane
1-(3-Dimethylaminopropyl)silantriol
(2-Dimethylaminoethyl)triethoxysilan.
(2-Dimethylaminoethyl)trimethoxysilane and/or
1-(2-Dimethylaminoethyl)silantriol.

The organic silicon compound of formula (I) is commercially available. (3-aminopropyl)trimethoxysilane, for example, can be purchased from Sigma-Aldrich. Also (3-aminopropyl)triethoxysilane is commercially available from Sigma-Aldrich.

In a further version, the invention contains at least one organic silicon compound of formula (II)

(R₅O)_{c}(R₆)_{d}Si(A)ₑ-[NR₇-(A′)]_{f}-[O-(A″)]_{g}-[NR₈-(A′″)]ₕ-Si(R₆′)_{d′}(OR₅′)_{c′} (II).

The organosilicon compounds of formula (II) according to the invention each carry the silicon-containing groups (R₅O)_{c}(R₆)_{d}Si— and —Si(R₆′)_{d}(OR₅′)_{c} at both ends.

In the central part of the molecule of formula (II) there are the groups -(A)ₑ- and —[NR₇-(A′)]_{f}- and —[O-(A″)]_{g}- and —[NR₈-(A′″)]ₕ-. Here, each of the radicals e, f, g, and h can independently of one another stand for the number 0 or 1, with the proviso that at least one of the radicals e, f, g, and h is different from 0. In other words, an organic silicon compound of formula (II) according to the invention contains at least one grouping from the group consisting of -(A)- and —[NR₇-(A′)]- and —[O-(A″)]- and —[NR₈-(A′″)]-.

In the two terminal structural units (R₅O)_{c}(R₆)_{d}Sii- and —Si(R₆′)_{d′}(OR₅′)_{c}, the radicals R5, R5′, R5″ independently of one another represent a hydrogen atom or a C₁-C₆ alkyl group. The radicals R6, R6′ and R6″ independently represent a C₁-C₆ alkyl group.

Here a stands for an integer from 1 to 3, and d stands for the integer 3−c. If c stands for the number 3, then d is equal to 0. If c stands for the number 2, then d is equal to 1. If c stands for the number 1, then d is equal to 2.

Analogously c′ stands for a whole number from 1 to 3, and d′ stands for the whole number 3−c′. If c′ stands for the number 3, then d′ is 0. If c′ stands for the number 2, then d′ is 1. If c′ stands for the number 1, then d′ is 2.

Dyeing's with the best wash fastness values could be obtained if the residues c and c′ both stand for the number 3. In this case d and d′ both stand for the number 0.

In another preferred embodiment, a process according to the invention is characterized in that the agent (a) contains at least one organic silicon compound of formula (II),

(R₅O)_{c}(R₆)_{d}Si-(A)ₑ-[NR₇-(A′)]_{f}-[O-(A″)]_{g}-[NR₈-(A′″)]ₕ-Si(R_{6′})_{d′}(OR₅′)_{c′} (II),

where
R5 and R5′ independently represent a methyl group or an ethyl group,
c and c′ both stand for the number 3 and
d and d′ both stand for the number 0.

If c and c′ are both the number 3 and d and d′ are both the number 0, the organic silicon compound of the invention corresponds to formula (IIa)

(R₅O)₃Si-(A)ₑ-[NR₇-(A′)]_{f}-[O-(A″)]_{g}-[NR₈-(A′″)]ₕ—Si(OR₅′)₃ (IIa).

The radicals e, f, g, and h can independently stand for the number 0 or 1, whereby at least one radical from e, f, g, and h is different from zero. The abbreviations e, f, g, and h thus define which of the groupings -(A)e- and —[NR7-(A′)]f- and —[O-(A″)]g- and —[NR8-(A′″)]h- are in the middle part of the organic silicon compound of formula (II).

In this context, the presence of certain groupings has proved to be particularly beneficial in terms of increasing washability. Particularly good results were obtained when at least two of the residues e, f, g, and h stand for the number 1. Especially preferred e and f both stand for the number 1. Furthermore, g and h both stand for the number 0.

If e and f both stand for the number 1 and g and h both stand for the number 0, the organic silicon compound according to the invention corresponds to formula (IIb)

(R₅O)_{c}(R₆)_{d}Si-(A)-[NR₇-(A′)]-Si(R₆′)_{d′}(OR₅′)_{c′} (IIb).

The radicals A, A′, A″, A′″ and A″″ independently represent a linear or branched divalent C₁-C₂₀ alkylene group. Preferably the radicals A, A′, A″, A′″ and A″″ independently of one another represent a linear, divalent C₁-C₂₀ alkylene group. Further preferably the radicals A, A′, A″, A′″ and A″″ independently represent a linear divalent C₁-C₆ alkylene group. In particular, the radicals A, A′, A″, A′″ and A″″ independently of one another represent a methylene group (—CH₂—), an ethylene group (—CH₂—CH₂—), a propylene group (—CH₂—CH₂—CH₂—) or a butylene group (—CH₂—CH₂—CH₂—CH₂—). In particular, the residues A, A′, A″, A′″ and A″″ stand for a propylene group (—CH₂—CH₂—CH₂—).

If the radical f represents the number 1, then the organic silicon compound of formula (II) according to the invention contains a structural grouping —[NR₇-(A′)]-.

If the radical f represents the number 1, then the organic silicon compound of formula (II) according to the invention contains a structural grouping —[NR₈-(A″)]-.

Wherein R₇ and R₇ independently represent a hydrogen atom, a C₁-C₆ alkyl group, a hydroxy-C₁-C₆ alkyl group, a C₂-C₆ alkenyl group, an amino-C₁-C₆ alkyl group or a group of the formula (III)

-(A″″)-Si(R₆″)_{d}″(OR₅″)_{c}″ (III).

Very preferably the radicals R7 and R8 independently of one another represent a hydrogen atom, a methyl group, a 2-hydroxyethyl group, a 2-alkenyl group, a 2-aminoethyl group or a grouping of the formula (III).

If the radical f represents the number 1 and the radical h represents the number 0, the organic silicon compound according to the invention contains the grouping [NR₇-(A′)] but not the grouping —[NR₈-(A′″)]. If the radical R7 now stands for a grouping of the formula (III), the pretreatment agent (a) contains an organic silicon compound with 3 reactive silane groups.

In another preferred embodiment, a process according to the invention is characterized in that the agent (a) contains at least one organic silicon compound of formula (II),

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),

where
e and f both stand for the number 1,
g and h both stand for the number 0,
A and A′ independently represent a linear, divalent C₁-C₆ alkylene group
   and
R7 represents a hydrogen atom, a methyl group, a 2-hydroxyethyl group, a 2-alkenyl group, a 2-aminoethyl group or a group of formula (III).

In a further preferred embodiment, a process according to the invention is characterized in that the agent (a) contains at least one organic silicon compound of the formula (II), where
e and f both stand for the number 1,
g and h both stand for the number 0,
A and A′ independently of one another represent a methylene group (—CH₂—), an ethylene group (—CH₂—CH₂—) or a propylene group (—CH₂—CH₂—CH₂), and
R7 represents a hydrogen atom, a methyl group, a 2-hydroxyethyl group, a 2-alkenyl group, a 2-aminoethyl group or a group of formula (III).

Organic silicon compounds of the formula (II) which are well suited for solving the problem according to the invention are
3-(trimethoxysilyl)-N-[3-(trimethoxysilyl)propyl]-1-propanamine

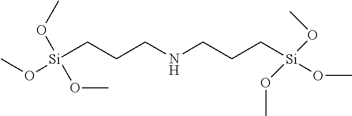

3-(Triethoxysilyl)-N-[3-(triethoxysilyl)propyl]-1-propanamine

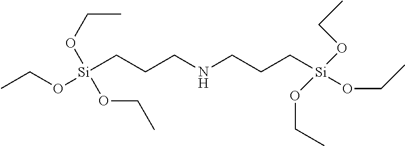 [SMILES: CCO[Si](CCCNCCC[Si](OCC)(OCC)OCC)(OCC)OCC]

N-methyl-3-(trimethoxysilyl)-N-[3-(trimethoxysilyl)propyl]-1-propanamine

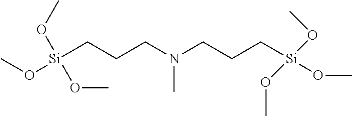

N-Methyl-3-(triethoxysilyl)-N-[3-(triethoxysilyl)propyl]-1-propanamine

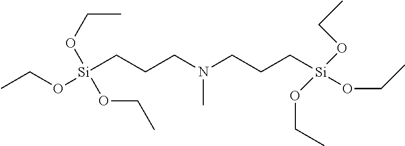 [SMILES: CCO[Si](CCCN(C)CCC[Si](OCC)(OCC)OCC)(OCC)OCC]

2-[Bis[3-(trimethoxysilyl)propyl]amino]-ethanol

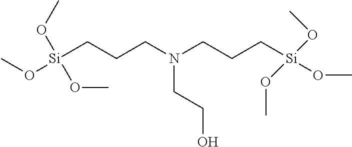

2-[bis[3-(triethoxysilyl)propyl]amino]ethanol

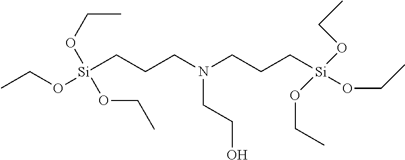 [SMILES: CCO[Si](CCCN(CCO)CCC[Si](OCC)(OCC)OCC)(OCC)OCC]

3-(Trimethoxysilyl)-N,N-bis[3-(trimethoxysilyl)propyl]-1-propanamine

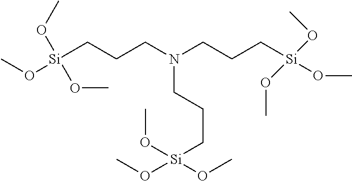

3-(Triethoxysilyl)-N,N-bis[3-(triethoxysilyl)propyl]-1-propanamine

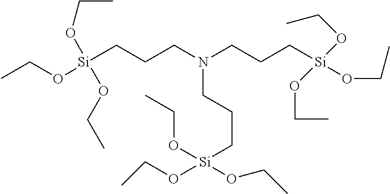 [SMILES: CCO[Si](CCCN(CCC[Si](OCC)(OCC)OCC)CCC[Si](OCC)(OCC)OCC)(OCC)OCC]

N1,N1-Bis[3-(trimethoxysilyl)propyl]-1,2-ethanediamine,

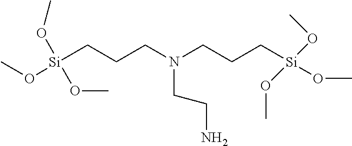

N1,N1-Bis[3-(triethoxysilyl)propyl]-1,2-ethanediamine,

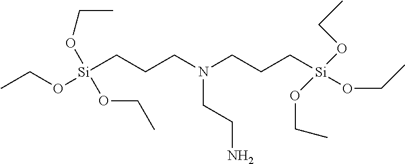 [SMILES: CCO[Si](CCCN(CCN)CCC[Si](OCC)(OCC)OCC)(OCC)OCC]

N,N-Bis[3-(trimethoxysilyl)propyl]-2-propen-1-amine

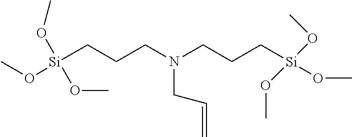

N,N-Bis[3-(triethoxysilyl)propyl]-2-propen-1-amine

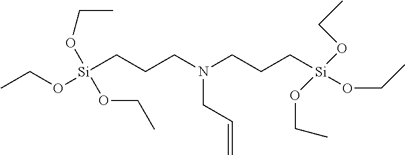 [SMILES: C=CCN(CCC[Si](OCC)(OCC)OCC)CCC[Si](OCC)(OCC)OCC]

The organic silicon compounds of formula (II) are commercially available.

Bis(trimethoxysilylpropyl)amines with the CAS number 82985-35-1 can be purchased from Sigma-Aldrich.

Bis[3-(triethoxysilyl)propyl]amines with the CAS number 13497-18-2 can be purchased from Sigma-Aldrich, for example.

N-methyl-3-(trimethoxysilyl)-N-[3-(trimethoxysilyl)propyl]-1-propanamine is alternatively referred to as bis(3-trimethoxysilylpropyl)-N-methylamine and can be purchased commercially from Sigma-Aldrich or Fluorochem.

3-(triethoxysilyl)-N,N-bis[3-(triethoxysilyl)propyl]-1-propanamine with the CAS number 18784-74-2 can be purchased for example from Fluorochem or Sigma-Aldrich.

In another preferred embodiment, a process according to the invention is characterized in that the agent (a) contains at least one organic silicon compound selected from the group consisting of
3-(trimethoxysilyl)-N-[3-(trimethoxysilyl)propyl]-1-propanamine
3-(Triethoxysilyl)-N-[3-(triethoxysilyl)propyl]-1-propanamine
N-methyl-3-(trimethoxysilyl)-N-[3-(trimethoxysilyl)propyl]-1-propanamine
N-Methyl-3-(triethoxysilyl)-N-[3-(triethoxysilyl)propyl]-1-propanamine
2-[Bis[3-(trimethoxysilyl)propyl]amino]-ethanol
2-[bis[3-(triethoxysilyl)propyl]amino]ethanol
3-(Trimethoxysilyl)-N,N-bis[3-(trimethoxysilyl)propyl]-1-propanamine
3-(Triethoxysilyl)-N,N-bis[3-(triethoxysilyl)propyl]-1-propanamine
N1,N1-bis[3-(trimethoxysilyl)propyl]-1,2-ethanediamine,
N1,N1-bis[3-(triethoxysilyl)propyl]-1,2-ethanediamine,
N,N-bis[3-(trimethoxysilyl)propyl]-2-propen-1-amine and/or
N,N-bis[3-(triethoxysilyl)propyl]-2-propen-1-amine.

In further dyeing tests, it has also proved to be particularly advantageous if the agent used on the keratinous material in the process according to the invention (a) contains at least one organic silicon compound of formula (IV)

R₉Si(OR₁₀)ₖ(R₁₁)ₘ (IV).

The compounds of formula (IV) are organic silicon compounds selected from silanes having one, two or three silicon atoms, the organic silicon compound comprising one or more hydroxyl groups and/or hydrolysable groups per molecule.

The organic silicon compound(s) of formula (IV) may also be called a silane of the alkyl-alkoxy-silane or alkyl-hydroxy-silane type,

R₉Si(OR₁₀)ₖ(R₁₁)ₘ (IV),

where
R₉ represents a C₁-C₁₂ alkyl group,
R₁₀ represents a hydrogen atom or a C₁-C₆ alkyl group,
R₁₁ represents a C₁-C₆ alkyl group
k is an integer from 1 to 3, and
m stands for the integer 3−k.

In another preferred embodiment, a process according to the invention is characterized in that the agent (a) contains at least one organic silicon compound of formula (IV).

R₉Si(OR₁₀)ₖ(R₁₁)ₘ (IV),

where
R₉ represents a C₁-C₁₂ alkyl group,
R₁₀ represents a hydrogen atom or a C₁-C₆ alkyl group,
R₁₁ represents a C₁-C₆ alkyl group
k is an integer from 1 to 3, and
m stands for the integer 3−k.

In a further preferred embodiment, a process according to the invention is characterized in that the agent (a) contains, in addition to the organic silicon compound(s) of formula (I), at least one further organic silicon compound of formula (IV)

R₉Si(OR₁₀)ₖ(R₁₁)ₘ (IV),

where
R₉ represents a C₁-C₁₂ alkyl group,
R₁₀ represents a hydrogen atom or a C₁-C₆ alkyl group,
R₁₁ represents a C₁-C₆ alkyl group
k is an integer from 1 to 3, and
m stands for the integer 3−k.

In a further preferred embodiment, a process according to the invention is characterized in that the agent (a) contains, in addition to the organic silicon compound(s) of formula (II), at least one further organic silicon compound of formula (IV)

R₉Si(OR₁₀)ₖ(R₁₁)ₘ (IV),

where
R₉ represents a C1-C12 alkyl group,
R₁₀ represents a hydrogen atom or a C₁-C₆ alkyl group,
R₁₁ represents a C₁-C₆ alkyl group
k is an integer from 1 to 3, and
m stands for the integer 3−k.

In a further preferred embodiment, a process according to the invention is characterized in that the composition contains (a) in addition to the organic silicon compound(s) of formula (I) and/or (II) at least one further organic silicon compound of formula (IV)

R₉Si(OR₁₀)ₖ(R₁₁)ₘ (IV),

where
R₉ represents a C1-C12 alkyl group,
R₁₀ represents a hydrogen atom or a C₁-C₆ alkyl group,
R₁₁ represents a C₁-C₆ alkyl group
k is an integer from 1 to 3, and
m stands for the integer 3−k.

In the organic silicon compounds of formula (IV), the radical R₉ represents a C₁-C₁₂ alkyl group. This C₁-C₁₂ alkyl group is saturated and can be linear or branched. Preferably R9 stands for a linear C₁-C₈ alkyl group. Preferably R₉ stands for a methyl group, an ethyl group, an n-propyl group, an n-butyl group, an n-pentyl group, an n-hexyl group, an n-octyl group or an n-dodecyl group. Particularly preferred, R9 stands for a methyl group, an ethyl group or an n-octyl group.

In the organic silicon compounds of formula (IV), the radical R10 represents a hydrogen atom or a C₁-C₆ alkyl group. R10 stands for a methyl group or an ethyl group.

In the organic silicon compounds of formula (IV), the radical R₁₁ represents a C₁-C₆ alkyl group. R11 stands for a methyl group or an ethyl group.

Furthermore, k stands for a whole number from 1 to 3, and m stands for the whole number 3−k. If k stands for the number 3, then m is equal to 0. If k stands for the number 2, then m is equal to 1. If k stands for the number 1, then m is equal to 2.

Dyes with the best wash fastness values could be obtained if an agent (a) were used in the process which contains at least one organic silicon compound of the formula (IV) in which the radical k stands for the number 3. In this case the rest m stands for the number 0.

Organic silicon compounds of the formula (IV) which are particularly suitable for solving the problem according to the invention are
Methyltrimethoxysilane

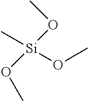

Methyltriethoxysilane

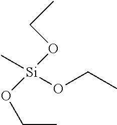

Ethyltrimethoxysilane

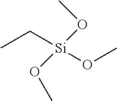

Ethyltriethoxysilane

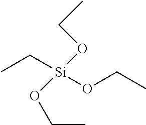

n-Hexyltrimethoxysilane

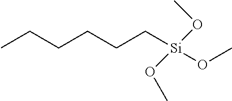

n-Hexyltriethoxysilane

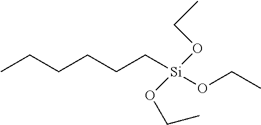 [SMILES: CCCCCC[Si](OCC)(OCC)OCC]

n-Octyltrimethoxysilane

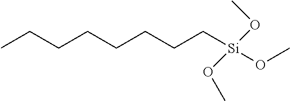

n-Octyltriethoxysilane

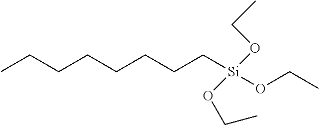 [SMILES: CCCCCCCC[Si](OCC)(OCC)OCC]

n-dodecyltrimethoxysilane and/or

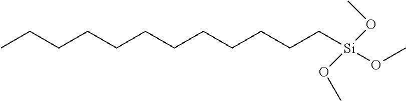 [SMILES: CCCCCCCCCCCC[Si](OC)(OC)OC]

n-dodecyltriethoxysilane.

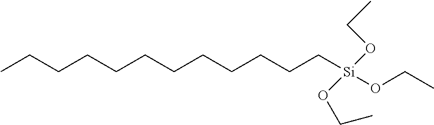 [SMILES: CCCCCCCCCCCC[Si](OCC)(OCC)OCC]

In another preferred embodiment, a process according to the invention is characterized in that the agent (a) contains at least one organic silicon compound of formula (IV) selected from the group consisting of
Methyltrimethoxysilane
Methyltriethoxysilane
Ethyltrimethoxysilane
Ethyltriethoxysilane
Hexyltrimethoxysilane
Hexyltriethoxysilane
Octyltrimethoxysilane
Octyltriethoxysilane
Dodecyltrimethoxysilane and/or
Dodecyltriethoxysilane.

The organic silicon compounds described above are reactive compounds. In this context, it has been found to be preferred if the agent (a) of the invention—based on the total weight of agent (a)—contains one or more organic silicon compounds in a total amount of 0.1 to 20.0% by weight, preferably 1.0 to 15.0% by weight and particularly preferably 2.0 to 8.0% by weight.

In a further preferred embodiment, a process according to the invention is characterized in that the agent (a)—based on the total weight of agent (a)—contains one or more organic silicon compounds in a total amount of 0.1 to 20.0% by weight, preferably 1.0 to 15.0% by weight and particularly preferably 2.0 to 8.0% by weight.

To achieve particularly good dyeing results, it is particularly advantageous to use the organic silicon compounds of the formula (I) and/or (II) in certain quantity ranges on average (a). Particularly preferably, the composition (a) contains—based on the total weight of composition (a)—one or more organic silicon compounds of the formula (I) and/or (II) in a total amount of 0.1 to 10.0% by weight, preferably 0.5 to 5.0% by weight and particularly preferably 0.5 to 3.0% by weight.

In a further preferred embodiment, a process according to the invention is characterized in that the agent (a)—based on the total weight of agent (a)—contains one or more organic silicon compounds of formula (I) and/or (II) in a total amount of 0.1 to 10.0% by weight, preferably 0.5 to 5.0% by weight and particularly preferably 0.5 to 3.0% by weight.

Furthermore, it has proven to be particularly preferred if the organic silicon compound(s) of formula (IV) is (are) also present in certain quantity ranges in average (a). Particularly preferably the agent (a) contains—based on the total weight of agent (a)—one or more organic silicon compounds of the formula (IV) in a total amount of 0.1 to 20.0% by weight, preferably 2.0 to 15.0% by weight and particularly preferably 4.0 to 9.0% by weight.

In a further preferred embodiment, a process according to the invention is characterized in that the agent (a)—based on the total weight of agent (a)—contains one or more organic silicon compounds of formula (IV) in a total amount of 0.1 to 20.0% by weight, preferably 2.0 to 15.0% by weight and particularly preferably 3.2 to 10.0% by weight.

In the course of the work leading to this invention it turned out that particularly stable and uniform films could be obtained on the keratin material if the agent (a) contains two structurally different organic silicon compounds.

In another preferred embodiment, a process according to the invention is characterized in that the agent (a) contains at least two structurally different organic silicon compounds.

In an explicitly particularly preferred embodiment, a process according to the invention is characterized in that an agent (a) is applied to the keratinous material which contains at least one organic silicon compound of the formula (I) which is selected from the group consisting of (3-aminopropyl)triethoxysilane and (3-aminopropyl)trimethoxysilane, and additionally contains at least one organic silicon compound of the formula (IV) which is selected from the group consisting of methyltrimethoxysilane, methyltriethoxysilane, ethyltrimethoxysilane and ethyltriethoxysilane.

In a further preferred embodiment, a process according to the invention is characterized in that the agent (a)—based on the total weight of agent (a)—contains:
0.5 to 3.0 weight % of at least one first organic silicon compound selected from the group of (3-aminopropyl)trimethoxysilane, (3-aminopropyl)triethoxysilane, (2-aminoethyl)trimethoxysilane, (2-aminoethyl)triethoxysilane, (3-dimethylaminopropyl)trimethoxysilane, (3-dimethylaminopropyl)triethoxysilane (2-dimethylaminoethyl)trimethoxysilane and (2-dimethylaminoethyl)triethoxysilane, and
3.2 to 10.0% by weight of at least one second organic silicon compound selected from the group of methyltrimethoxysilane, methyltriethoxysilane, ethyltrimethoxysilane, ethyltriethoxysilane, octyltrimethoxysilane, octyltriethoxysilane, dodecyltrimethoxysilane and dodecyltriethoxysilane.

In this version, the agent contains (a) one or more organic silicon compounds of a first group in a total amount of 0.5 to 3.0% by weight. The organic silicon compounds of this first group are selected from the group of (3-aminopropyl)trimethoxysilane, (3-aminopropyl)triethoxysilane, (2-aminoethyl)trimethoxysilane, (2-aminoethyl)triethoxysilane, (3-dimethylaminopropyl)trimethoxysilane, (3-dimethylaminopropyl)triethoxysilane (2-dimethylaminoethyl)trimethoxysilane and/or (2-dimethylaminoethyl)triethoxysilane.

In this version, the agent contains (a) one or more organic silicon compounds of a second group in a total amount of 3.2 to 10.0% by weight. The organic silicon compounds of this second group are selected from the group of methyltrimethoxysilane, methyltriethoxysilane, ethyltrimethoxysilane, ethyltriethoxysilane, octyltrimethoxysilane, octyltriethoxysilane, dodecyltrimethoxysilane and/or dodecyltriethoxysilane.

### Surfactants

As the second essential component, the agent used in the process according to the invention (a) contains at least one surfactant. The use of cationic and/or non-ionic surfactants has proven to be particularly preferred.

In a further preferred embodiment, a process according to the invention is characterized in that the agent (a) contains at least one surfactant from the group of cationic and/or non-ionic surfactants.

Within the scope of a design, the agent used in the process according to the invention (a) contains at least one cationic surfactant.

Cationic surfactants are surfactants, i.e., surface-active compounds, each with one or more positive charges. Cationic surfactants contain only positive charges. Usually, these surfactants are composed of a hydrophobic part and a hydrophilic head group, the hydrophobic part usually consisting of a hydrocarbon backbone (e.g., consisting of one or two linear or branched alkyl chains) and the positive charge(s) being in the hydrophilic head group. Cationic surfactants adsorb at interfaces and aggregate in aqueous solution above the critical micelle formation concentration to form positively charged micelles.

Examples of cationic surfactants are
quaternary ammonium compounds, which may carry one or two alkyl chains with a chain length of 8 to 28 carbon atoms as hydrophobic residues
quaternary phosphonium salts substituted by one or more alkyl chains with a chain length of 8 to 28 carbon atoms or
tertiary sulfonium salts.

Furthermore, the cationic charge can also be part of a heterocyclic ring (e.g., an imidazolium ring or a pyridinium ring) in the form of an onium structure.

In addition to the functional unit that carries the cationic charge, the cationic surfactant may also contain other uncharged functional groups, as is the case with esterquats.

Particularly uniform films could be obtained if an agent (a) containing at least one cationic surfactant of formula (X) was used in the process according to the invention,

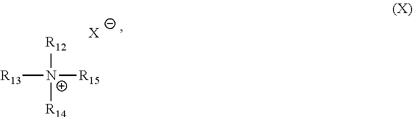

wherein
R12, R13, R14 are independently a C₁-C₆ alkyl group, a C₂-C₆ alkenyl group or a C₂-C₆ hydroxyalkyl group,
R15 represents a C₈-C₂₈ alkyl group and
X− stands for a physiologically compatible anion.

In a further preferred embodiment, a process according to the invention is characterized in that the agent (a) contains at least one cationic surfactant of formula (X),

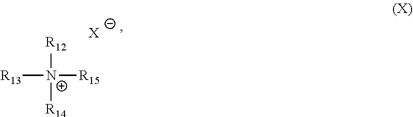

wherein
R12, R13, R14 are independently a C₁-C₆ alkyl group, a C₂-C₆ alkenyl group or a C₂-C₆ hydroxyalkyl group,
R15 represents a C₈-C₂₈ alkyl group and
X− stands for a physiologically compatible anion.

The radicals R12, R13 and R14 independently of one another represent a C₁-C₆ alkyl group, a C₂-C₆ alkenyl group or a C₂-C₆ hydroxyalkyl group.

Preferably, R12, R13 and R14 independently represent a methyl group, an ethyl group, or a 2-hydroxyethyl group. R12, R13 and R14 are particularly preferred methyl groups.

The radical R15 represents a C₈-C₂₈ alkyl group. Preferably R15 represents a C₁₂ alkyl group, a C₁₄ alkyl group, a C₁₆ alkyl group, a C₁₈ alkyl group, a C₂₀ alkyl group or a C₂₂ alkyl group. The above-mentioned alkyl groups are preferably linear. R15 stands for a linear C₁₆ alkyl group, a linear C₁₈ alkyl group or a linear C₂₀ alkyl group.

A particularly preferred cationic surfactant of formula (X) is, for example, cetrimonium chloride (trimethyl hexadecyl ammonium chloride), which can be obtained from Clariant under the trade name Genamin CTAC, or from BASF under the trade name Dehyquart A-CA.

Furthermore, it is also particularly preferred if the agent used in the process according to the invention (a) contains at least one imidazolium compound as cationic surfactant.

Within the group of cationic imidazolium compounds, compounds of the formula (XI) have proven to be particularly compatible with the organic silicon compounds

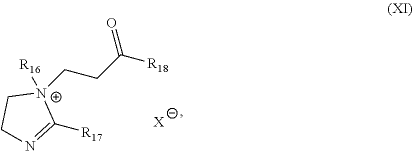

wherein
R16 represents a C₁-C₆ alkyl group
R17, R18 independently of one another represent a C₇-C₂₇ alkyl group and
X− stands for a physiologically compatible anion.

Using the cationic imidazolium compounds (XI) in agent (a), it was also possible to obtain very intense and uniform colors.

In a further preferred embodiment, a process according to the invention is characterized in that the agent (a) contains at least one cationic surfactant of formula (XI),

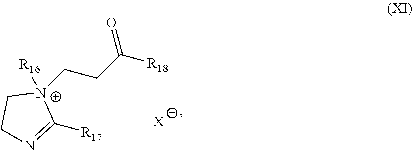

wherein
R16 represents a C₁-C₆ alkyl group
R17, R18 independently of one another represent a C₇-C₂₇ alkyl group and
X− stands for a physiologically compatible anion.

Keratinic fibers could be dyed in intensive nuances if, in addition to the organic silicon compounds, at least one cationic surfactant of the formula (XI) was used in the agents (a) according to the invention, in which

R16 stands for a methyl group,
R17, R18 independently of one another represent a C8-C27 alkyl group and
X⁻ stands for a physiologically compatible anion.

Especially preferred compounds of formula (XI) are known under the INCI name Quaternium-87, which are also sold under the trade names Varisoft W 575 PB or Revoquat PG 75.

Particularly intensive staining results could also be achieved if one or more compounds from the esterquats group were used as cationic surfactants on average (a). The name esterquats is a collective term for cationic surface-active compounds with two hydrophobic groups, which are linked via ester bonds to a quaternized di- or triethanolamine or an analogous compound.

Esterquats are known substances that contain at least one ester function and at least one quaternary ammonium group as structural element. Preferred esterquats are quaternized ester salts of fatty acids with triethanolamine, quaternized ester salts of fatty acids with diethanolalkylamines and quaternized ester salts of fatty acids with 1.2-dihydroxypropyl dialkylamines. Such products are sold under the trademarks Stepantex®, Dehyquart® and Armocare®. The products Armocare® VGH-70, N,N-Bis(2-Palmitoyloxyethyl)dimethylammonium chloride, and Dehyquart® F-75, Dehyquart® C-4046, Dehyquart® L80 and Dehyquart® AU-35 and Dehyquart AU-35 are examples of such esterquats.

Especially well suited esterquats are the compounds of formula (XII),

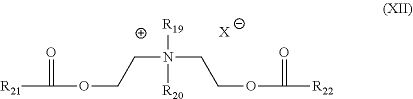

wherein
R19, R20 are independently a C₁-C₆ alkyl group or a C₂-C₆ hydroxyalkyl group,
R21, R22 independently of one another represent a C₇-C₂₇ alkyl group and
X− stands for a physiologically compatible anion.

In a further preferred embodiment, a process according to the invention is characterized in that the agent (a) contains at least one cationic surfactant de formula (XII),

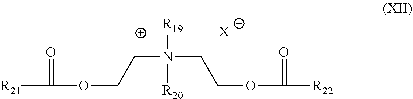

wherein
R19, R20 are independently a C₁-C₆ alkyl group or a C₂-C₆ hydroxyalkyl group,
R21, R22 independently of one another represent a C₇-C₂₇ alkyl group and
X− stands for a physiologically compatible anion.

In another particularly preferred embodiment, a process according to the invention is characterized in that the agent (a) contains one or more compounds of formula (XII),

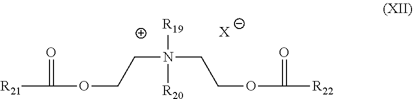

wherein
R19, R20 independently represent a methyl group, an ethyl group, a propyl group, or a 2-hydroxyethyl group,
   R21, R22 independently of one another represent a C15 alkyl group or a C17 alkyl group and
   X⁻ stands for a physiologically compatible anion.

In formulas (X) to (XII), X⁻ stands for a physiologically compatible anion. Suitable physiologically compatible anions are halide (especially chloride or bromide), hydrogen sulfate, sulfate, benzenesulfonate, p-toluenesulfonate, acetate, citrate, lactate, tartrate, methyl sulfate (H₃COSO₃⁻, methylsulfocant or trifluoromethanesulfonate. A- stands for chloride, bromide or for methyl sulfate (H3COSO3-).

The agent (a) used in the process according to the invention contains the cationic surfactant(s) preferably within certain quantity ranges.

In a further particularly preferred embodiment, a process according to the invention is therefore characterized in that the agent (a)—based on the total weight of agent (a)—contains one or more cationic surfactants (b) in a total amount of from 0.1 to 10.0% by weight, preferably from 0.2 to 8.0% by weight, more preferably from 0.3 to 6.0% by weight and particularly preferably from 0.4 to 4.0% by weight.

In a further version, the agent used in the process according to the invention (a) contains at least one non-ionic surfactant.

Nonionic surfactants are surfactants that do not have charges but strong dipole moments and are strongly hydrated in aqueous solution.

Particularly well-suited nonionic surfactants contain as hydrophilic group e.g., a polyol group, a polyalkylene glycol ether group or a combination of polyol and polyglycol ether group. Such links include
Addition products of 2 to 50 mol ethylene oxide and/or 0 to 5 mol propylene oxide to linear and branched fatty alcohols with 6 to 30 C atoms, the fatty alcohol polyglycol ethers or the fatty alcohol polypropylene glycol ethers or mixed fatty alcohol polyethers,
Addition products of 2 to 50 mol ethylene oxide and/or 0 to 5 mol propylene oxide to linear and branched fatty acids with 6 to 30 C atoms, the fatty acid polyglycol ethers or the fatty acid polypropylene glycol ethers or mixed fatty acid polyethers,
Addition products of 2 to 50 mol ethylene oxide and/or 0 to 5 mol propylene oxide to linear and branched alkylphenols having 8 to 15 C atoms in the alkyl group, the alkylphenol polyglycol ethers or the alkylpolypropylene glycol ethers or mixed alkylphenol polyethers,
with a methyl or C₂-C₆-alkyl radical end-group capped addition products of 2 to 50 moles of ethylene oxide and/or 0 to 5 moles of propylene oxide to linear and branched fatty alcohols with 8 to 30 C atoms, to fatty acids with 8 to 30 C atoms and to alkylphenols with 8 to 15 C atoms in the alkyl group, such as the grades available under the sales names Dehydol® LS, Dehydol® LT (Cognis),
C12-C30 fatty acid mono- and diesters of addition products of 1 to 30 mol ethylene oxide to glycerol,
Addition products of 5 to 60 mol ethylene oxide to castor oil and hardened castor oil,
Polyol fatty acid esters, such as the commercial product Hydagen® HSP (Cognis) or Sovermol® grades (Cognis),
alkoxylated triglycerides,
alkoxylated fatty acid alkyl esters of the formula (Tnio-1)

R¹CO—(OCH₂CHR²)_{w}OR³ (Tnio-1)
in which R¹CO is a linear or branched, saturated and/or unsaturated acyl radical having 6 to 22 carbon atoms, R2 is hydrogen or methyl, R³ is linear or branched alkyl radicals having 1 to 4 carbon atoms and w is numbers from 1 to 20,
amine oxides,
Hydroxy mixed ethers, as described for example in DE-OS 19738866,
Sorbitan fatty acid esters and addition products of ethylene oxide to sorbitan fatty acid esters such as polysorbates,
Sugar fatty acid esters and addition products of ethylene oxide to sugar fatty acid ester,
Addition products of ethylene oxide to fatty acid alkanolamides and fatty amines,
Sugar tensides of the alkyl and alkenyl oligoglycoside type according to formula (E4-II),

R⁴O-[G]ₚ (Tnio-2)

in which R⁴ is an alkyl or alkenyl radical containing 4 to 22 carbon atoms, G is a sugar residue containing 5 or 6 carbon atoms and p is several 1 to 10. They can be obtained by the relevant methods of preparative organic chemistry. The alkyl and alkenyl oligoglycosides can be derived from aldoses or ketoses with 5 or 6 carbon atoms, preferably glucose. The preferred alkyl and/or alkenyl oligoglycosides are thus alkyl and/or alkenyl oligoglucosides. The index number p in the general formula (Tnio-2) indicates the degree of oligomerization (DP), i.e., the distribution of mono- and oligoglycosides and stands for a number between 1 and 10. While p must always be an integer in the individual molecule and can assume the values p=1 to 6, the value p for a certain alkyl oligoglycoside is an analytically determined arithmetical quantity, which usually represents a fractional number. Preferably alkyl and/or alkenyl oligoglycosides with an average degree of oligomerization p of 1.1 to 3.0 are used. From an application technology point of view, those alkyl and/or alkenyl oligoglycosides are preferred whose degree of oligomerization is less than 1.7 and lies between 1.2 and 1.4. The alkyl or alkenyl radical R⁴ can be derived from primary alcohols containing 4 to 11, preferably 8 to 10 carbon atoms. Typical examples are butanol, caproic alcohol, caprylic alcohol, caprin alcohol and undecrylic alcohol as well as their technical mixtures, such as those obtained in the hydrogenation of technical fatty acid methyl esters or during the hydrogenation of aldehydes from Roelen's oxo synthesis. Preferred are alkyl oligoglucosides with a chain length of C₈-C₁₀ (DP=1 to 3), which are obtained as a preliminary step in the distillative separation of technical C₈-C₁₈ coconut-fatty alcohol and may be contaminated with less than 6% by weight of C₁₂ alcohol, and alkyl oligoglucosides based on technical C_{9/11} oxoalcohols (DP=1 to 3). The alkyl or alkenyl radical R¹⁵ can also be derived from primary alcohols having 12 to 22, preferably 12 to 14 carbon atoms. Typical examples are lauryl alcohol, myristyl alcohol, cetyl alcohol, palmoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, brassidyl alcohol and their technical mixtures, which can be obtained as described above. Preferred are alkyl oligoglucosides based on hardened C12/14 coconut alcohol with a DP of 1 to 3.
Sugar surfactants of the fatty acid N-alkyl polyhydroxyalkylamide type, a nonionic surfactant of formula (Tnio-3)

R⁵CO—NR⁶—[Z] (Tnio-3)

in which R⁵CO is an aliphatic acyl radical containing 6 to 22 carbon atoms, R⁶ is hydrogen, an alkyl or hydroxyalkyl radical containing 1 to 4 carbon atoms and [Z] is a linear or branched polyhydroxyalkyl radical containing 3 to 12 carbon atoms and 3 to 10 hydroxyl groups. The fatty acid N-alkyl polyhydroxyalkylamides are known substances that can usually be obtained by reductive amination of a reducing sugar with ammonia, an alkylamine or an alkanolamine and subsequent acylation with a fatty acid, a fatty acid alkyl ester or a fatty acid chloride. The fatty acid N-alkyl polyhydroxyalkylamides are preferably derived from reducing sugars with 5 or 6 carbon atoms, especially from glucose. The preferred fatty acid N-alkyl polyhydroxyalkylamides are therefore fatty acid N-alkylglucamides as represented by the formula (Tnio-4):

R⁷CO—(NR⁸)—CH₂—[CH(OH)]₄—CH₂OH (Tnio-4)

Preferably, glucamides of the formula (Tnio-4) are used as fatty acid-N-alkyl polyhydroxyalkylamides, in which R⁸ represents hydrogen or an alkyl group and R⁷CO represents the acyl radical of caproic acid, caprylic acid, capric acid, Lauric acid, myristic acid, palmitic acid, palmoleic acid, stearic acid, isostearic acid, oleic acid, elaidic acid, petroselinic acid, linoleic acid, linolenic acid, arachidic acid, gadoleic acid, behenic acid or erucic acid or their technical mixtures. Particularly preferred are fatty acid N-alkyl glucamides of the formula (Tnio-4), which are obtained by reductive amination of glucose with methylamine and subsequent acylation with lauric acid or C12/14 coconut fatty acid or a corresponding derivative. Furthermore, polyhydroxyalkylamides can also be derived from maltose and palatinose.

The agent (a) used in the process according to the invention contains the non-ionic surfactant(s) preferably within certain quantity ranges.

In a further particularly preferred embodiment, a process according to the invention is therefore characterized in that the agent (a)—based on the total weight of agent (a)—contains one or more nonionic surfactants in a total amount of from 0.1 to 10.0% by weight, preferably from 0.2 to 8.0% by weight, more preferably from 0.3 to 6.0% by weight and particularly preferably from 0.4 to 4.0% by weight.

### Agent (b)

The agent (b) is characterized by its content of at least one pigment. The agent (b) may also be called colorant (b).

Pigments within the meaning of the present invention are coloring compounds which have a solubility in water at 25° C. of less than 0.5 g/L, preferably less than 0.1 g/L, even more preferably less than 0.05 g/L. Water solubility can be determined, for example, by the method described below: 0.5 g of the pigment are weighed in a beaker. A stir-fish is added. Then one liter of distilled water is added. This mixture is heated to 25° C. for one hour while stirring on a magnetic stirrer. If undissolved components of the pigment are still visible in the mixture after this period, the solubility of the pigment is below 0.5 g/L. If the pigment-water mixture cannot be assessed visually due to the high intensity of the possibly finely dispersed pigment, the mixture is filtered. If a proportion of undissolved pigments remains on the filter paper, the solubility of the pigment is below 0.5 g/L.

Suitable color pigments can be of inorganic and/or organic origin.

In a preferred embodiment, an agent (b) of the invention is characterized in that it contains at least one colorant compound from the group of inorganic and/or organic pigments.

Preferred color pigments are selected from synthetic or natural inorganic pigments. Inorganic color pigments of natural origin can be produced, for example, from chalk, ochre, umber, green earth, burnt Terra di Siena or graphite. Furthermore, black pigments such as iron oxide black, colored pigments such as ultramarine or iron oxide red as well as fluorescent or phosphorescent pigments can be used as inorganic color pigments.

Particularly suitable are colored metal oxides, hydroxides and oxide hydrates, mixed-phase pigments, sulfur-containing silicates, silicates, metal sulfides, complex metal cyanides, metal sulphates, chromates and/or molybdates. Preferred color pigments are black iron oxide (CI 77499), yellow iron oxide (CI 77492), red and brown iron oxide (CI 77491), manganese violet (CI 77742), ultramarine (sodium aluminum sulfo silicates, CI 77007, pigment blue 29), chromium oxide hydrate (CI77289), iron blue (ferric ferrocyanides, 0177510) and/or carmine (cochineal).

According to the invention, colored pearlescent pigments are also particularly preferred color pigments. These are usually mica- and/or mica-based and can be coated with one or more metal oxides. Mica belongs to the layer silicates. The most important representatives of these silicates are muscovite, phlogopite, paragonite, biotite, lepidolite and margarite. To produce the pearlescent pigments in combination with metal oxides, the mica, mainly muscovite or phlogopite, is coated with a metal oxide.

As an alternative to natural mica, synthetic mica coated with one or more metal oxides can also be used as pearlescent pigment. Especially preferred pearlescent pigments are based on natural or synthetic mica (mica) and are coated with one or more of the metal oxides mentioned above. The color of the respective pigments can be varied by varying the layer thickness of the metal oxide(s).

In a further preferred embodiment, a process according to the invention is characterized in that the agent (b) contains at least one colorant compound from the group of pigments selected from the group of colored metal oxides, metal hydroxides, metal oxide hydrates, silicates, metal sulfides, complex metal cyanides, metal sulfates, bronze pigments and/or from colored pigments based on mica or mica which are coated with at least one metal oxide and/or one metal oxychloride.

In a further preferred embodiment, an agent according to the invention is characterized in that it contains (b) at least one colorant compound from the group of pigments selected from pigments based on mica or micaceous iron oxide, which is combined with one or more metal oxides from the group of titanium dioxide (CI 77891), are coated with black iron oxide (CI 77499), yellow iron oxide (CI 77492), red and/or brown iron oxide (CI 77491, CI 77499), manganese violet (CI 77742), ultramarine (sodium aluminum sulfo silicates, CI 77007, pigment blue 29), chromium oxide hydrate (CI 77289), chromium oxide (CI 77288) and/or iron blue (ferric ferrocyanides, CI 77510).

Examples of particularly suitable color pigments are commercially available under the trade names Rona®, Colorona®, Xirona®, Dichrona® and Timiron® from Merck, Ariabel® and Unipure® from Sensient, Prestige® from Eckart Cosmetic Colors and Sunshine® from Sunstar.

Particularly preferred color pigments with the trade name Colorona® are, for example:

### Colorona Copper, Merck, MICA, CI 77491 (IRON OXIDES)

### Colorona Passion Orange, Merck, Mica, CI 77491 (Iron Oxides), Alumina

### Colorona Patina Silver, Merck, MICA, CI 77499 (IRON OXIDES), CI 77891 (TITANIUM DIOXIDE)

### Colorona RY, Merck, CI 77891 (TITANIUM DIOXIDE), MICA, CI 75470 (CARMINE)

### Colorona Oriental Beige, Merck, MICA, CI 77891 (TITANIUM DIOXIDE), CI 77491 (IRON OXIDES)

### Colorona Dark Blue, Merck, MICA, TITANIUM DIOXIDE, FERRIC FERROCYANIDE

### Colorona Chameleon, Merck, CI 77491 (IRON OXIDES), MICA

### Colorona Aborigine Amber, Merck, MICA, CI 77499 (IRON OXIDES), CI 77891 (TITANIUM DIOXIDE)

### Colorona Blackstar Blue, Merck, CI 77499 (IRON OXIDES), MICA

### Colorona Patagonian Purple, Merck, MICA, CI 77491 (IRON OXIDES), CI 77891 (TITANIUM DIOXIDE), CI 77510 (FERRIC FERROCYANIDE)

### Colorona Red Brown, Merck, MICA, CI 77491 (IRON OXIDES), CI 77891 (TITANIUM DIOXIDE)

### Colorona Russet, Merck, CI 77491 (TITANIUM DIOXIDE), MICA, CI 77891 (IRON OXIDES)

### Colorona Imperial Red, Merck, MICA, TITANIUM DIOXIDE (CI 77891), D&C RED NO. 30 (CI 73360)

### Colorona Majestic Green, Merck, CI 77891 (TITANIUM DIOXIDE), MICA, CI 77288 (CHROMIUM OXIDE GREENS)

### Colorona Light Blue, Merck, MICA, TITANIUM DIOXIDE (CI 77891), FERRIC FERROCYANIDE (CI 77510)

### Colorona Red Gold, Merck, MICA, CI 77891 (TITANIUM DIOXIDE), CI 77491 (IRON OXIDES)

### Colorona Gold Plus MP 25, Merck, MICA, TITANIUM DIOXIDE (CI 77891), IRON OXIDES (CI 77491)

### Colorona Carmine Red, Merck, MICA, TITANIUM DIOXIDE, CARMINE

### Colorona Blackstar Green, Merck, MICA, CI 77499 (IRON OXIDES)

### Colorona Bordeaux, Merck, MICA, CI 77491 (IRON OXIDES)

### Colorona Bronze, Merck, MICA, CI 77491 (IRON OXIDES)

### Colorona Bronze Fine, Merck, MICA, CI 77491 (IRON OXIDES)

### Colorona Fine Gold MP 20, Merck, MICA, CI 77891 (TITANIUM DIOXIDE), CI 77491 (IRON OXIDES)

### Colorona Sienna Fine, Merck, CI 77491 (IRON OXIDES), MICA

### Colorona Sienna, Merck, MICA, CI 77491 (IRON OXIDES)

Colorona Precious Gold, Merck, Mica, CI 77891 (Titanium dioxide), Silica, CI 77491 (Iron oxides), Tin oxide

### Colorona Sun Gold Sparkle MP 29, Merck, MICA, TITANIUM DIOXIDE, IRON OXIDES, MICA, CI 77891, CI 77491 (EU)

Colorona Mica Black, Merck, CI 77499 (Iron oxides), Mica, CI 77891 (Titanium dioxide)
Colorona Bright Gold, Merck, Mica, CI 77891 (Titanium dioxide), CI 77491 (Iron oxides)

### Colorona Blackstar Gold, Merck, MICA, CI 77499 (IRON OXIDES)

Other particularly preferred color pigments with the trade name Xirona® are for example:

### Xirona Golden Sky, Merck, Silica, CI 77891 (Titanium Dioxide), Tin Oxide

### Xirona Caribbean Blue, Merck, Mica, CI 77891 (Titanium Dioxide), Silica, Tin Oxide

### Xirona Kiwi Rose, Merck, Silica, CI 77891 (Titanium Dioxide), Tin Oxide

### Xirona Magic Mauve, Merck, Silica, CI 77891 (Titanium Dioxide), Tin Oxide.

In addition, particularly preferred color pigments with the trade name Unipure® are for example:

### Unipure Red LC 381 EM, Sensient CI 77491 (Iron Oxides), Silica

### Unipure Black LC 989 EM, Sensient, CI 77499 (Iron Oxides), Silica

### Unipure Yellow LC 182 EM, Sensient, CI 77492 (Iron Oxides), Silica

In a further embodiment, the means according to the invention may also contain (b) one or more coloring compounds from the group of organic pigments

The organic pigments according to the invention are correspondingly insoluble, organic dyes or color lacquers, which may be selected, for example, from the group of nitroso, nitro-azo, xanthene, anthraquinone, isoindolinone, isoindolinone, quinacridone, perinone, perylene, diketo-pyrrolopyorrole, indigo, thioindido, dioxazine and/or triarylmethane compounds.

Examples of particularly suitable organic pigments are carmine, quinacridone, phthalocyanine, sorghum, blue pigments with the Color Index numbers CI 42090, CI 69800, CI 69825, CI 73000, CI 74100, CI 74160, yellow pigments with the Color Index numbers CI 11680, CI 11710, CI 15985, CI 19140, CI 20040, CI 21100, CI 21108, CI 47000, CI 47005, green pigments with the Color Index numbers CI 61565, CI 61570, CI 74260, orange pigments with the Color Index numbers CI 11725, CI 15510, CI 45370, CI 71105, red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

In a further particularly preferred embodiment, a process according to the invention is characterized in that the agent (b) contains at least one colorant compound from the group of organic pigments selected from the group of carmine, quinacridone, phthalocyanine, sorghum, blue pigments with the Color Index numbers CI 42090, CI 69800, CI 69825, CI 73000, CI 74100, CI 74160, yellow pigments with the Color Index numbers CI 11680, CI 11710, CI 15985, CI 19140, CI 20040, CI 21100, CI 21108, CI 47000, CI 47005, green pigments with Color Index numbers CI 61565, CI 61570, CI 74260, orange pigments with Color Index numbers CI 11725, CI 15510, CI 45370, CI 71105, red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

The organic pigment can also be a color paint. In the sense of the invention, the term color lacquer means particles comprising a layer of absorbed dyes, the unit of particle and dye being insoluble under the above-mentioned conditions. The particles can, for example, be inorganic substrates, which can be aluminum, silica, calcium borosilate, calcium aluminum borosilicate or even aluminum.

For example, alizarin color varnish can be used.

Due to their excellent light and temperature resistance, the use of the pigments in agent (b) of the process according to the invention is particularly preferred. It is also preferred if the pigments used have a certain particle size. This particle size leads on the one hand to an even distribution of the pigments in the formed polymer film and on the other hand avoids a rough hair or skin feeling after application of the cosmetic product. According to the invention, it is therefore advantageous if the at least one pigment has an average particle size D50 of 1.0 to 50 μm, preferably 5.0 to 45 μm, preferably 10 to 40 μm, 14 to 30 μm. The mean particle size D50D₅₀, for example, can be determined using dynamic light scattering (DLS).

The pigment or pigments may be used in an amount of 0.001 to 20% by weight, of 0.05 to 5% by weight, each based on the total weight of agent (b).

### Film Forming, Hydrophobic Polymer

To achieve uniform and particularly washfast dyeing's, it has further proved to be particularly preferred if the agent (b) used in the process according to the invention additionally contains at least one film-forming, hydrophobic polymer.

In a further preferred embodiment, a process according to the invention is characterized in that agent (b) contains at least one film-forming, hydrophobic polymer.

Polymers are macromolecules with a molecular weight of at least 1000 g/mol, preferably of at least 2500 g/mol, particularly preferably of at least 5000 g/mol, which consist of identical, repeating organic units. The polymers of the present invention may be synthetically produced polymers which are manufactured by polymerization of one type of monomer or by polymerisation of different types of monomer which are structurally different from each other. If the polymer is produced by polymerizing a type of monomer, it is called a homo-polymer. If structurally different monomer types are used in polymerisation, the resulting polymer is called a copolymer.

The maximum molecular weight of the polymer depends on the degree of polymerisation (number of polymerized monomers) and the batch size and is determined by the polymerisation method. For the purposes of the present invention, it is preferred that the maximum molecular weight of the film-forming hydrophobic polymer (c) is not more than 107 g/mol, preferably not more than 106 g/mol and particularly preferably not more than 105 g/mol.

A hydrophobic polymer is a polymer that has a solubility in water at 25° C. (760 mmHg) of less than 1% by weight.

The water solubility of the film-forming, hydrophobic polymer can be determined in the following way, for example. 1.0 g of the polymer is placed in a beaker. Make up to 100 g with water. A stir-fish is added, and the mixture is heated to 25° C. on a magnetic stirrer while stirring. It is stirred for 60 minutes. The aqueous mixture is then visually assessed. If the polymer-water mixture cannot be assessed visually due to a high turbidity of the mixture, the mixture is filtered. If a proportion of undissolved polymer remains on the filter paper, the solubility of the polymer is less than 1% by weight.

In the sense of the invention, a film-forming polymer is a polymer which can form a film on a substrate, for example on a keratinic material or a keratinic fiber. The formation of a film can be demonstrated, for example, by looking at the keratin material treated with the polymer under a microscope.

These include acrylic acid-type polymers, polyurethanes, polyesters, polyamides, polyureas, cellulose polymers, nitrocellulose polymers, silicone polymers, acrylamide-type polymers, and polyisoprenes.

Particularly well suited film-forming, hydrophobic polymers are, for example, polymers from the group of copolymers of acrylic acid, copolymers of methacrylic acid, homopolymers or copolymers of acrylic acid esters, homopolymers or copolymers of methacrylic acid esters, homopolymers or copolymers of acrylic acid amides, homopolymers or copolymers of methacrylic acid amides, copolymers of vinylpyrrolidone, copolymers of vinyl alcohol, copolymers of vinyl acetate, homopolymers or copolymers of ethylene, homopolymers or copolymers of propylene, homopolymers or copolymers of styrene, polyurethanes, polyesters and/or polyamides.

In a further preferred embodiment, a process according to the invention is characterized in that the agent (b) contains at least one film-forming hydrophobic polymer selected from the group of copolymers of acrylic acid, copolymers of methacrylic acid, homopolymers or copolymers of acrylic acid esters, homopolymers or copolymers of methacrylic acid esters, the homopolymers or copolymers of acrylic acid amides, the homopolymers or copolymers of methacrylic acid amides, the copolymers of vinylpyrrolidone, the copolymers of vinyl alcohol, the copolymers of vinyl acetate, the homopolymers or copolymers of ethylene, the homopolymers or copolymers of propylene, the homopolymers or copolymers of styrene, polyurethanes, polyesters and/or polyamides

The film-forming hydrophobic polymers, which are selected from the group of synthetic polymers, polymers obtainable by radical polymerisation or natural polymers, have proved to be particularly suitable for solving the problem according to the invention.

Other particularly well-suited film-forming hydrophobic polymers can be selected from the homopolymers or copolymers of olefins, such as cycloolefins, butadiene, isoprene or styrene, vinyl ethers, vinylamides, the esters or amides of (meth)acrylic acid with at least one C₁-C₂₀ alkyl group, an aryl group or a C2-C10 hydroxyalkyl group.

Other film-forming hydrophobic polymers may be selected from the homo- or copolymers of isooctyl (meth)acrylate; isonononyl (meth)acrylate; 2-ethylhexyl (meth)acrylate; lauryl (meth)acrylate; isopentyl (meth)acrylate; n-butyl (meth)acrylate); isobutyl (meth)acrylate; ethyl (meth)acrylate; methyl (meth)acrylate; tert-butyl (meth)acrylate; stearyl (meth)acrylate; hydroxyethyl (meth)acrylate; 2-hydroxypropyl (meth)acrylate; 3-hydroxypropyl (meth)acrylate and/or mixtures thereof.

Other film-forming hydrophobic polymers may be selected from the homo- or copolymers of (meth)acrylamide; N-alkyl-(meth)acrylamides, in those with C2-C18 alkyl groups, such as N-ethyl-acrylamide, N-tert-butyl-acrylamide, le N-octyl-crylamide; N-di(C1-C4)alkyl-(meth)acrylamide.

Other preferred anionic copolymers are, for example, copolymers of acrylic acid, methacrylic acid or their C₁-C₆ alkyl esters, as they are marketed under the INCI Declaration Acrylates Copolymers. A suitable commercial product is for example Aculyn® 33 from Rohm & Haas. Copolymers of acrylic acid, methacrylic acid or their C₁-C₆ alkyl esters and the esters of an ethylenically unsaturated acid and an alkoxylated fatty alcohol are also preferred. Suitable ethylenically unsaturated acids are especially acrylic acid, methacrylic acid and itaconic acid; suitable alkoxylated fatty alcohols are especially steareth-20 or ceteth-20.

Some of the most preferred polymers on the market are Aculyn 22 (Acrylates/Steareth-20 Methacrylate Copolymer), Aculyn 28 (Acrylates/Beheneth-25 Methacrylate Copolymer), Structure 2001® (Acryla-tes/Steareth-20 Itaconate Copolymer), Structure 3001® (Acrylates/Ceteth-20 Itaconate Copolymer), Structure Plus (Acrylates/Aminoacrylates 010-30 Alkyl PEG-20 Itaconate Copolymer), Carbopol® 1342, 1382, Ultrez 20, Ultrez 21 (Acrylates/C10-30 Alkyl Acrylates Crosspolymer), Synthalen W 2000® (Acrylates/Palmeth-25 Acrylates Copolymer) or the Soltex OPT (Acrylates/C12-22 Alkyl Methacrylate Copolymer) distributed by Rohme and Haas.

The homo- and copolymers of N-vinylpyrrolidone, vinylcaprolactam, vinyl-(C1-C6)alkyl-pyrrole, vinyl-oxazole, vinyl-thiazole, vinylpyrimidine, vinylimidazole can be named as suitable polymers based on vinyl monomers.

Furthermore, the copolymers octylacrylamide/acrylates/ butylaminoethyl-methacrylate copolymer, as commercially marketed under the trade names AMPHOMER® or LOVOCRYL® 47 by NATIONAL STARCH, or the copolymers of acrylates/octylacrylamides marketed under the trade names DERMACRYL® LT and DERMACRYL® 79 by NATIONAL STARCH are particularly suitable.

Suitable olefin-based polymers include homopolymers and copolymers of ethylene, propylene, butene, isoprene and butadiene.

In another version, block copolymers can be used as film-forming hydrophobic polymers, which comprise at least one block of styrene or the derivatives of styrene. These block copolymers can be copolymers that contain one or more other blocks in addition to a styrene block, such as styrene/ethylene, styrene/ethylene/butylene, styrene/butylene, styrene/isoprene, styrene/butadiene. Such polymers are commercially distributed by BASF under the trade name “Luvitol HSB”.

Surprisingly, it turned out that particularly good dyeing's can be obtained with the anionic direct dyes, if the film-forming hydrophobic polymer also carries anionic charges.

In a further explicitly particularly preferred embodiment, a process according to the invention is characterized in that the agent (b) contains at least one anionic, film-forming, hydrophobic polymer.

An anionic polymer is a polymer comprising repeating units having at least one carboxylic acid group, one sulphonic acid group and/or their physiologically acceptable salts. In other words, an anionic polymer is made from monomers having at least one carboxylic acid group, a sulphonic acid group. In this context, the hydrophobic, film-forming copolymers of acrylic acid and the copolymers of methacrylic acid are particularly preferred. The polymers in this group contain the carboxylic acid groups, the sulphonic acid groups or their salts in an amount that ensures that the hydrophobic character of the whole polymer is maintained.

The film-forming hydrophobic polymer(s) according to the invention are preferably used in certain quantity ranges on average (b). In this context, it has proved to be particularly preferred for the solution of the task according to the invention if the agent (b)—based on the total weight of agent (b)—contains one or more polymers in a total amount of 0.1 to 25.0% by weight, preferably from 0.2 to 20.0% by weight, more preferably from 0.5 to 15.0% by weight and very particularly preferably from 1.0 to 7.0% by weight.

In a further preferred embodiment, a process according to the invention is characterized in that agent (b) contains—based on the total weight of agent (b)—one or more film-forming hydrophobic polymers in a total amount of from 0.1 to 25.0% by weight, preferably from 0.2 to 20.0% by weight, more preferably from 0.5 to 15.0% by weight and very particularly preferably from 1.0 to 7.0% by weight.

### Other Ingredients in Products (a) and (b)

The agents (a) and (b) described above may also contain one or more optional ingredients.

The agents (especially agent (b)) may additionally contain one or more surfactants. The term surfactants refer to surface-active substances. A distinction is made between anionic surfactants consisting of a hydrophobic residue and a negatively charged hydrophilic head group, amphoteric surfactants, which carry both a negative and a compensating positive charge, cationic surfactants, which in addition to a hydrophobic residue have a positively charged hydrophilic group, and non-ionic surfactants, which have no charges but strong dipole moments and are strongly hydrated in aqueous solution.

Zwitterionic surfactants are those surface-active compounds which carry at least one quaternary ammonium group and at least one —COO⁽⁻⁾— or —SO₃⁽⁻⁾ group in the molecule. Particularly suitable zwitterionic surfactants are the so-called betaines such as the N-alkyl-N,N-dimethylammonium-glycinate, for example the cocoalkyl-dimethylammoniumglycinate, N-acylaminopropyl-N,N-dimethylammoniumglycinate, for example, cocoacylaminopropyl dimethyl ammonium glycinate, and 2-alkyl-3-carboxymethyl-3-hydroxyethyl imidazolines each having 8 to 18 C atoms in the alkyl or acyl group, and cocoacylaminoethyl hydroxyethyl carboxymethyl glycinate. A preferred zwitterionic surfactant is the fatty acid amide derivative known under the INCI name cocamidopropyl betaine.

Ampholytic surfactants are surface-active compounds which, apart from a C₈-C₂₄ alkyl or acyl group, contain at least one free amino group and at least one —COOH— or —SO₃H group in the molecule and can form internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids each with about 8 to 24 C atoms in the alkyl group. Typical examples of amphoteric or zwitterionic surfactants are alkylbetaines, alkylamidobetaines, amino propionates, aminoglycinate, imidazoliniumbetaines and sulfobetaines.

Particularly preferred ampholytic surfactants are N-cocoalkylaminopropionate, cocoacylaminoethylaminopropionate and C₁₂-C₁₈ acylsarcosine.

The products may also additionally contain at least one non-ionic surfactant. Suitable non-ionic surfactants are alkyl polyglycosides as well as alkylene oxide addition products to fatty alcohols and fatty acids with 2 to 30 mol ethylene oxide per mol fatty alcohol or fatty acid. Preparations with good properties are also obtained if they contain as non-ionic surfactants fatty acid esters of ethoxylated glycerol reacted with at least 2 mol ethylene oxide. The non-ionic surfactants are used in a total quantity of 0.1 to 45% by weight, preferably 1 to 30% by weight and very preferably 1 to 15% by weight—based on the total weight of the respective agent.

In addition, the products may also contain at least one cationic surfactant. Cationic surfactants are surfactants, i.e., surface-active compounds, each with one or more positive charges. Cationic surfactants contain only positive charges. Usually, these surfactants are composed of a hydrophobic part and a hydrophilic head group, the hydrophobic part usually consisting of a hydrocarbon backbone (e.g., consisting of one or two linear or branched alkyl chains) and the positive charge(s) being in the hydrophilic head group. Examples of cationic surfactants are
quaternary ammonium compounds which, as hydrophobic radicals, may carry one or two alkyl chains with a chain length of 8 to 28 C atoms,
quaternary phosphonium salts substituted with one or more alkyl chains with a chain length of 8 to 28 C atoms or
tertiary sulfonium salts.

Furthermore, the cationic charge can also be part of a heterocyclic ring (e.g., an imidazolium ring or a pyridinium ring) in the form of an onium structure. In addition to the functional unit carrying the cationic charge, the cationic surfactant may also contain other uncharged functional groups, as is the case for example with esterquats. The cationic surfactants are used in a total quantity of 0.1 to 45 wt. %, preferably 1 to 30 wt. % and most preferably 1 to 15 wt. %—based on the total weight of the respective agent.

Furthermore, the means according to the invention may also contain at least one anionic surfactant. Anionic surfactants are surface-active agents with exclusively anionic charges (neutralized by a corresponding counter cation). Examples of anionic surfactants are fatty acids, alkyl sulphates, alkyl ether sulphates and ether carboxylic acids with 12 to 20 C atoms in the alkyl group and up to 16 glycol ether groups in the molecule.

The anionic surfactants are used in a total quantity of 0.1 to 45 wt. %, preferably 1 to 30 wt. % and most preferably 1 to 15 wt. %—based on the total weight of the respective agent.

To adjust the desired pH value, agents (a) and (b) may also contain at least one alkalizing agent and/or acidifying agent. The pH values for the purposes of the present invention are pH values measured at a temperature of 22° C.

As alkalizing agents, agents (a), (b) and (c) may contain for example ammonia, alkanolamines and/or basic amino acids.

The alkanolamines which can be used in the composition of the invention are preferably selected from primary amines having a C₂-C₆ alkyl base which carries at least one hydroxyl group. Preferred alkanolamines are selected from the group formed by 2-aminoethan-1-ol (monoethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropan-1,2-diol, 2-amino-2-methylpropan-1,3-diol.

Alkanolamines particularly preferred according to the invention are selected from 2-aminoethan-1-ol and/or 2-amino-2-methylpropan-1-ol. A particularly preferred embodiment is therefore characterized in that the agent according to the invention contains an alkanolamine selected from 2-aminoethan-1-ol and/or 2-amino-2-methylpropan-1-ol as alkalizing agent.

A particularly preferred embodiment is therefore characterized in that the agent according to the invention contains an alkanolamine selected from 2-aminoethan-1-ol and/or 2-amino-2-methylpropan-1-ol as alkalizing agent. Preferred amino acids are amino carboxylic acids, especially α-(alpha)-amino carboxylic acids and w-amino carboxylic acids, whereby α-amino carboxylic acids are particularly preferred.

According to the invention, basic amino acids are those amino acids which have an isoelectric point pI of greater than 7.0.

Basic α-amino carboxylic acids contain at least one asymmetric carbon atom. In the context of the present invention, both possible enantiomers can be used equally as specific compounds or their mixtures, especially as racemates. However, it is particularly advantageous to use the naturally preferred isomeric form, usually in L-configuration.

The basic amino acids are preferably selected from the group formed by arginine, lysine, ornithine, and histidine, especially preferably arginine and lysine. In another particularly preferred embodiment, an agent according to the invention is therefore characterized in that the alkalizing agent is a basic amino acid from the group arginine, lysine, ornithine and/or histidine.

In addition, the product may contain other alkalizing agents, especially inorganic alkalizing agents. Inorganic alkalizing agents usable according to the invention are preferably selected from the group formed by sodium hydroxide, potassium hydroxide, calcium hydroxide, barium hydroxide, sodium phosphate, potassium phosphate, sodium silicate, sodium metasilicate, potassium silicate, sodium carbonate and potassium carbonate.

Particularly preferred alkalizing agents are ammonia, 2-aminoethan-1-ol (monoethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-Amino-2-methylpropan-2-ol, 3-aminopropan-1,2-diol, 2-amino-2-methylpropan-1,3-diol, arginine, lysine, ornithine, histidine, sodium hydroxide, potassium hydroxide, calcium hydroxide, barium hydroxide, sodium phosphate, potassium phosphate, sodium silicate, sodium metasilicate, potassium silicate, sodium carbonate and potassium carbonate.

Acidifiers commonly used by experts are, for example, indulgence acids such as citric acid, acetic acid, malic acid, or tartaric acid, as well as diluted mineral acids such as hydrochloric acid, sulfuric acid, or phosphoric acid.

They may also contain other active substances, auxiliaries and additives, such as solvents, fatty components such as C₈-C₃₀ fatty alcohols, C₈-C₃₀ fatty acid triglycerides, C₈-C₃₀ fatty acid monoglycerides, 08-030 fatty acid diglycerides and/or hydrocarbons; polymers, structural agents such as glucose, maleic acid and lactic acid; hair conditioning compounds such as phospholipids, for example lecithin and cephalins; perfume oils, dimethylisosorbide and cyclodextrins; fiber structure-improving active substances, in particular mono-, di- and oligosaccharides such as glucose, galactose, fructose, fructose and lactose; dyes for coloring the composition; anti-dandruff active substances such as Piroctone Olamine, Zinc Omadine and Climbazol; amino acids and oligopeptides; protein hydrolysates on animal and/or vegetable basis, as well as in the form of their fatty acid condensation products or optionally anionic or cationically modified derivatives; vegetable oils; sunscreens and UV-blockers; active ingredients such as panthenol, pantothenic acid, pantolactone, allantoin, pyrrolidinonecarboxylic acids and their salts, and bisabolol; polyphenols, in particular hydroxycinnamic acids, 6,7-dihydroxycumarine, hydroxybenzoic acids, catechin, tannine, leukoanthocyanidine, anthocyanidine, flavanone, flavone and flavonols; ceramides or pseudoceramides; vitamins, provitamins and vitamin precursors; plant extracts; fats and waxes such as fatty alcohols, beeswax, montan wax and paraffins; swelling and penetrating substances such as glycerol, propylene glycol monoethyl ether, carbonates, hydrogen carbonates, guanidines, ureas and primary, secondary and tertiary phosphates; opacifiers such as latex, styrene/PVP and styrene/acrylamide copolymers; pearlescent agents such as ethylene glycol mono- and distearate and PEG-3 distearate; and blowing agents such as propane-butane mixtures, N₂O, dimethyl ether, CO₂ and air.

The selection of these other substances will be made by the specialist according to the desired properties of the agents. About other optional components and the quantities of these components used, explicit reference is made to the relevant manuals known to the specialist. The additional active ingredients and auxiliary substances are preferably used in the preparations according to the invention in quantities of 0.0001 to 25 wt. % each, 0.0005 to 15 wt. %, based on the total weight of the respective agent.

### Process for Dyeing Keratin Materials

In the procedure according to the invention, agents (a) and (b) are applied to the keratinous materials, to human hair. Thus, agents (a) and (b) are the ready-to-use agents. The agents (a) and (b) are different.

In principle, agents (a) and (b) can be applied simultaneously or successively, whereby successive application is preferred.

The best results were obtained when agent (a) was applied to the keratin materials as a pretreatment agent and then agent (b) was applied as a colorant.

Therefore, a method for dyeing keratinous material, in particular human hair, comprising the following steps in the order given is particularly preferred:
in a first step, applying an agent (a) to the keratinous material, the agent (a) comprising at least one organic silicon compound and at least one surfactant, and
in a second step, applying an agent (b) to the keratinous material, the agent (b) containing at least one pigment,

The agents (a) and (b) are preferably used within one and the same dyeing process, which means that there is a maximum period of several hours between the application of agents (a) and (b). Especially preferred is a period of maximum 30 minutes between the application of agents (a) and (b).

In the context of a further preferred embodiment, a method according to the invention is characterized in that first the agent (a) is applied, then the agent (b) is applied, wherein the period between the application of the agents (a) and (b) is a maximum of 24 hours, preferably a maximum of 12 hours, further preferably a maximum of 6 hours and most preferably a maximum of 30 minutes.

Within the scope of the procedure according to the invention, the keratin materials, in particular human hair, are first treated with agent (a). Then the actual colorant (b)—which contains the coloring compounds—is applied to the keratin materials.

Preferably, agent (a) itself does not contain colorants or coloring compounds. The pre-treatment agent (a) is characterized by its content of at least one reactive organic silicon compound and at least one surfactant. The reactive organic silicon compound(s) (a) functionalize the hair surface as soon as they meet it. In this way a first, still uncolored film is formed. It has been found that this film is particularly uniform due to the presence of the surfactants in agent (a), which leads on the one hand to intensive colorations and on the other hand to a particularly uniform color result. In the second step of the process, a colorant (b) is now applied to the hair. During the application of colorant (b), the colorant pigments interact with the silane film and are thus bound to the keratin materials. Here, the technical application properties of the resulting dyeing can be further improved by selecting the optimum process conditions.

In the context of a further form of execution, a procedure comprising the following steps in the order indicated is particularly preferred

(1) Application of agent (a) on the keratinous material,
(2) Allow the agent (a) to act for a period of 10 seconds to 10 minutes, preferably from 10 seconds to 5 minutes,
(3) if necessary, rinse the keratinous material with water,
(4) Application of agent (b) on the keratinous material,
(5) Allow the agent (b) to act for a period of 30 seconds to 30 minutes, preferably from 30 seconds to 10 minutes, and
(6) Rinse the keratinous material with water.

The rinsing of the keratinous material with water in steps (3) and (6) of the process is understood, according to the invention, to mean that only water is used for the rinsing process, without any other agents other than agents (a) and (b).

In a first step (1), agent (a) is applied to the keratin materials, especially human hair.

After application, the agent (a) can act on the keratin materials. In this context, application times from 10 seconds to 10 minutes, preferably from 20 seconds to 5 minutes and especially preferably from 30 seconds to 2 minutes on the hair have proven to be particularly beneficial.

In a preferred embodiment of the method according to the invention, the agent (a) can now be rinsed from the keratin materials before the agent (b) is applied to the hair in the subsequent step.

Dyeing's with also good wash fastness were obtained when agent (b) was applied to the keratin materials which were still exposed to agent (a).

In step (4), agent (b) is now applied to the keratin materials. After application, let the agent (b) act on the hair.

The process according to the invention allows the production of dyeing's with particularly good intensity and wash fastness even with a short exposure time of agent (b). Application times from 10 seconds to 10 minutes, preferably from 20 seconds to 5 minutes and most preferably from 30 seconds to 3 minutes on the hair have proven to be particularly beneficial.

In step (6), agent (b) (and any remaining agent (a)) is rinsed out of the keratin material with water.

In the context of a further form of execution, a procedure comprising the following steps in the order indicated is particularly preferred

(1) Application of agent (a) on the keratinous material,
(2) Allow the agent (a) to act for a period of 10 seconds to 10 minutes, preferably from 10 seconds to 5 minutes,
(3) Rinse the keratinous material with water,
(4) Application of agent (b) on the keratinous material,
(5) Allow the agent (b) to act for a period of 30 seconds to 30 minutes, preferably from 30 seconds to 10 minutes, and
(6) Rinse the keratinous material with water.

In this design, the sequence of steps (1) to (6) preferably takes place within 24 hours, particularly preferably within 6 hours.

### Multi-Component Packaging Unit (Kit-of-Parts)

Within the scope of the procedure according to the invention, agents (a) and (b) are applied to the keratin materials, i.e., both agents (a) and (b) are ready-to-use agents.

To increase user comfort, the user is preferably provided with all required resources in the form of a multi-component packaging unit (kit-of-parts).

A second subject matter of the present invention is therefore a multi-component packaging unit (kit-of-parts) for coloring keratinic material, comprehensively packaged separately from one another
a first container containing an agent (a), said agent (a) comprising at least one organic silicon compound and at least one surfactant, as disclosed in detail in the description of the first article of invention, and
a second container containing an agent (b), wherein the agent (b) contains at least one pigment as disclosed in detail in the description of the first object of invention.

The organic silicon compounds contained in agent (a) of the kit correspond to the organic silicon compounds that were also used in agent (a) of the method described above.

The surfactants contained in agent (a) of the kit correspond to the surfactants that were also used in agent (a) of the procedure described above

The pigments contained in agent (b) of the kit correspond to the pigments that were also used in agent (b) of the procedure described above.

A second subject of the present invention is therefore a multi-component packaging unit (kit-of-parts) for coloring keratinous material, comprehensively packaged separately from one another
a first container containing an agent (a), said agent (a) comprising at least one organic silicon compound and at least one surfactant, said organic silicon compound and said surfactant having been disclosed in detail in the description of the first article of invention: and
a second container containing an agent (b), said agent (b) containing at least one pigment, said pigment having been disclosed in detail when the first article of invention was described.

The agent (a) contains with the organic silicon compound(s) a class of highly reactive compounds which can undergo hydrolysis or oligomerization and/or polymerization in the presence of water as described above. Due to their high reactivity, these organic silicon compounds form a film on the keratin material.

To avoid premature oligomerization or polymerization, it may be of considerable advantage to the user to prepare the ready-to-use agent (a) only shortly before application.

In the context of a further embodiment, a multi-component packaging unit (kit-of-parts) for coloring keratinic material is preferably packaged separately from one another
a first container with an agent (a1), wherein the agent (a1) contains at least one organic silicon compound and at least one surfactant, and
a second container with an agent (a2), wherein the agent (a2) contains water and
a third container containing an agent (b), the agent (b) containing at least one pigment.

In the context of a further embodiment, a multi-component packaging unit (kit-of-parts) for coloring keratinic material is preferably packaged separately from one another
a first container with an agent (a1′), wherein the agent (a1′) contains at least one organic silicon compound, and
a second container with an agent (a2′), the agent (a2′) containing at least one surfactant and water, and
a third container containing an agent (b), the agent (b) containing at least one pigment.

To provide a formulation that is as stable as possible during storage, the agent (al) itself is preferably packaged with low or no water.

A kit-of-parts packaging unit according to the invention is characterized in that the agent (a1)—based on the total weight of the agent (a1)—contains a water content of from 0.001 to 10.0% by weight, preferably from 0.5 to 9.0% by weight, more preferably from 1.0 to 8.0% by weight and very particularly preferably from 1.5 to 7.0% by weight.

The agent (a2) contains water. In a preferred embodiment, a multi-component packaging unit (kit-of-parts) according to the invention is characterized in that the agent (a2)—based on the total weight of the agent (a2)—has a water content of from 15 to 100% by weight, preferably from 35 to 100% by weight, more preferably from 55 to 100% by weight, still more preferably from 65 to 100% by weight and very particularly preferably from 75 to 100% by weight.

Within this version, the ready-to-use agent (a) is now produced by mixing agents (al) and (a2).

For example, the user can first mix or shake the agent (a1) containing the organic silicon compound(s) with the water-containing agent (a2). The user can now apply this mixture of (a1) and (a2) to the keratin materials—either directly after their production or after a short reaction time of 10 seconds to 20 minutes. Afterwards, the user can apply agent (b) as described above.

With respect to the other preferred embodiments of the multi-component packaging unit according to the invention, the same applies mutatis mutandis to the procedure according to the invention.

### EXAMPLES

### 1. Formulations

The following formulations were produced:

### Pretreatment Agent (a)

| Agent (a1) | (a11) | (a12) | (a13) | (a14) |
|---|---|---|---|---|
| (3-Aminopropyl)triethoxysilan | 13.3 g | 13.3 g | 13.3 g | 13.3 g |
| Methyltrimethoxysilane | 46.7 g | 46.7 g | 46.7 g | 46.7 g |
| Cetyltrimethylammonium chloride | 3.0 g | — | — | — |
| Quaternium-87 | — | 3.0 g | 1.5 g | — |
| Ceteareth-20 | — | — | 1.5 g | — |
| Water | 7.0 g | 7.0 g | 7.0 g | 10.0 g |

| Agent (a2) | (a21) | (a22) | (a23) | (a24) |
|---|---|---|---|---|
| Cetyltrimethylammonium | — | 3.0 g | — | — |
| chloride | | | | |
| Quaternium-87 | — | — | 3.0 g | 1.5 g |
| Ceteareth-20 | — | — | — | 1.5 g |
| Ammonia/citric acid | ad pH 9.5 | ad pH 9.5 | ad pH 9.5 | ad pH 9.5 |
| Water | 100 g | 100 g | 100 g | 100 g |

### Colorants

| Agent (b) | (b1) | (b2) |
|---|---|---|
| Colorona Bronze (Merck, Mica, | 2.0 | — |
| CI77491, Iron oxides, CI77019) | | |
| Colorona Red Gold, Merck, MICA, | — | 3.0 |
| CI 77891 (TITANIUM DIOXIDE), CI | | |
| 77491 (IRON OXIDES) | | |
| Dermacryl 79 (Akzo Nobel, | 9.0 | 9.0 |
| Acrylates/Octylacrylamide | | |
| Copolymer, CAS-Nr. 129702-02-9) | | |
| Ammonia (25% aqueous solution) | ad pH 10 | ad pH 10 |
| Water | ad 100 | ad 100 |

### 2. Application

To prepare the pre-treatment agent (a) ready for use, the indicated quantity of the stored agent (a1) was mixed with the indicated quantity of the agent (a2) while shaking. Afterwards the agent (a) was left to stand for 15 minutes. The agent (a) is the ready-to-use agent.

One strand of hair each (Kerling, dark brown) was dipped into the agent (a) and left there for 1 minute. Afterwards, excess product (a) was stripped from each strand of hair. Each strand of hair was washed out with water. Excess water was scraped off each strand of hair.

Immediately afterwards, the hair strands were each dipped into the agent (b) and left in it for 1 minute. Afterwards, excess agent (b) was stripped from each strand of hair. Then each strand of hair was washed thoroughly (1 minute) with water and dried.

| Specimen | 1 | 2 | 3 |
|---|---|---|---|
| Agent (a1) | (a11) | (a12) | (a13) |
| | 5.0 g | 5.0 g | 5.0 g |
| Agent (a2) | (a21) | (a21) | (a21) |
| | 5.0 g | 5.0 g | 5.0 g |
| Agent (b) | (b1) | (b1) | (b1) |

| Specimen | 4 | 5 | 6 |
|---|---|---|---|
| Agent (a1) | (a14) | (a14) | (a14) |
| | 5.0 g | 5.0 g | 5.0 g |
| Agent (a2) | (a22) | (a23) | (a24) |
| | 5.0 g | 5.0 g | 5.0 g |
| Agent (b) | (b2) | (b2) | (b2) |

In examples 1 to 6, very intensive and very uniform colorations were obtained.

In the case of examples 4, 5, and 6, a red-golden shimmering strand was obtained, which was lighter in comparison to the dark brown hair used.

1. Process for dyeing keratinous material, in particular human hair, comprising the following steps:
Application of an agent (a) to the keratinous material, the agent (a) containing at least one organic silicon compound and at least one surfactant, and
Application of an agent (b) to the keratinous material, the agent (b) containing at least one pigment.

2. Process according to claim 1, characterized in that the agent (a) contains at least one organic silicon compound of formula (I) and/or (II)
R₁R₂N-L-Si(OR₃)ₐ(R₄)_{b} (I)
where
R₁, R₂ independently represent a hydrogen atom or a C₁-C₆ alkyl group,
L is a linear or branched divalent C₁-C₂₀ alkylene group,
R₁, R₄ independently of one another represent a C₁-C₆ alkyl group,
a, stands for an integer from 1 to 3, and
b stands for the integer 3−a, and
wherein in the organic silicon compound of formula (II)
(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),
R5, R5′, R5″, R6, R6′ and R6″ independently represent a C₁-C₆ alkyl group,
A, A′, A″, A′″ and A″″ independently represent a linear or branched divalent C₁-C₂₀ alkylene group,
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III)
-(A″″)-Si(R₆″)_{d″}(OR_{5″})_{c″} (III),
c, stands for an integer from 1 to 3,
d stands for the integer 3−c,
c′ stands for an integer from 1 to 3,
d′ stands for the integer 3−c′,
c″ stands for an integer from 1 to 3,
d″ stands for the integer 3−c″,
e stands for 0 or 1,
f stands for 0 or 1,
g stands for 0 or 1,
h stands for 0 or 1,
provided that at least one of e, f, g, and h is different from 0.

3. Process according to one of claims 1 to 2, characterized in that the agent (a) contains at least one organic silicon compound of the formula (I),
R₁R₂N-L-Si(OR₃)ₐ(R₄)_{b} (I),
where
R₁, R₂ both represent a hydrogen atom, and
L represents a linear, divalent C₁-C₆-alkylene group, preferably a propylene group (—CH₂—CH₂—CH₂—) or an ethylene group (—CH₂—CH₂—),
R₃, R₄ independently of one another represent a methyl group or an ethyl group and
a stands for the number 3 and
b stands for the number 0.

4. Process according to any one of claims 1 to 3, characterized in that the agent (a) contains at least one organic silicon compound selected from the group consisting of
(3-Aminopropyl)trimethoxysilane
(3-Aminopropyl)triethoxysilan
(2-Aminoethyl)trimethoxysilane
(2-Aminoethyl)triethoxysilan
(3-Dimethylaminopropyl)trimethoxysilane
(3-Dimethylaminopropyl)triethoxysilan
(2-dimethylaminoethyl)trimethoxysilane and
(2-Dimethylaminoethyl)triethoxysilan.

5. Process according to any one of claims 1 to 4, characterized in that the agent (a) contains at least one organic silicon compound of the formula (IV),
R₉Si(OR₁₀)ₖ(R₁₁)ₘ (IV),
where
R₉ represents a C₁-C₁₂ alkyl group,
R₁₀ represents a hydrogen atom or a C₁-C₆ alkyl group,
R₁₁ represents a C₁-C₆ alkyl group
k is an integer from 1 to 3, and
m stands for the integer 3−k.

6. A process according to any one of claims 1 to 5, characterized in that the agent (a) comprises at least one organic silicon compound selected from the group consisting of
Methyltrimethoxysilane
Methyltriethoxysilane
Ethyltrimethoxysilane
Ethyltriethoxysilane
Octyltrimethoxysilane
Octyltriethoxysilane
Dodecyltrimethoxysilane and
Dodecyltriethoxysilane.

7. Process according to one of claims 1 to 6, characterized in that the agent (a) contains at least two structurally different organic silicon compounds.

8. Process according to one of claims 1 to 7, characterized in that the agent (a)—based on the total weight of agent (a)—contains:
0.5 to 3.0 weight % of at least one first organic silicon compound selected from the group of (3-aminopropyl)trimethoxysilane, (3-aminopropyl)triethoxysilane, (2-aminoethyl)trimethoxysilane, (2-aminoethyl)triethoxysilane, (3-dimethylaminopropyl)trimethoxysilane, (3-dimethylaminopropyl)triethoxysilane (2-dimethylaminoethyl)trimethoxysilane and (2-dimethylaminoethyl)triethoxysilane, and
3.2 to 10.0% by weight of at least one second organic silicon compound selected from the group of methyltrimethoxysilane, methyltriethoxysilane, ethyltrimethoxysilane, ethyltriethoxysilane, octyltrimethoxysilane, octyltriethoxysilane, dodecyltrimethoxysilane and dodecyltriethoxysilane.

9. A process according to any one of claims 1 to 8, characterized in that the composition (a) contains at least one surfactant from the group of nonionic and/or cationic surfactants.

10. Process according to any one of claims 1 to 9, characterized in that the agent (a) contains at least one cationic surfactant corresponding to formula (X), 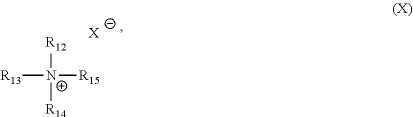
wherein
R12, R13, R14 are independently a C₁-C₆ alkyl group, a C₂-C₆ alkenyl group or a C₂-C₆ hydroxyalkyl group,
R15 represents a C₈-C₂₈ alkyl group and
X− stands for a physiologically compatible anion,

11. Process according to any one of claims 1 to 10, characterized in that the agent (a) contains at least one cationic surfactant corresponding to formula (XI), 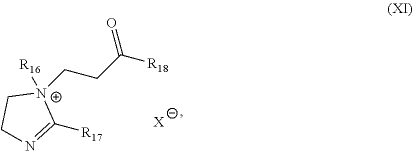
wherein
R16 represents a C₁-C₆ alkyl group
R17, R18 independently of one another represent a C₇-C₂₇ alkyl group and
X− stands for a physiologically compatible anion.

12. Process according to any one of claims 1 to 11, characterized in that the agent (a) contains at least one cationic surfactant de formula (XII), 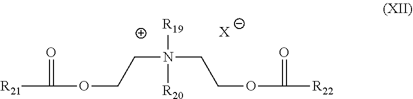
wherein
R19, R20 are independently a C₁-C₆ alkyl group or a C₂-C₆ hydroxyalkyl group,
R21, R22 independently of one another represent a C₇-C₂₇ alkyl group and
X− stands for a physiologically compatible anion.

13. A process according to one of claims 1 to 12, characterized in that the agent (b) at least one inorganic pigment selected from the group consisting of colored metal oxides, metal hydroxides, metal oxide hydrates, silicates, metal sulfides, complex metal cyanides, metal sulfates, bronze pigments and/or colored pigments based on mica or mica coated with at least one metal oxide and/or one metal oxychloride.

14. A process according to one of claims 1 to 13, characterized in that the agent (b) contains at least one organic pigment selected from the group consisting of carmine, quinacridone, phthalocyanine, sorghum, blue pigments with the Color Index numbers CI 42090, CI 69800, CI 69825, CI 73000, CI 74100, CI 74160, yellow pigments with the Color Index numbers CI 11680, CI 11710, CI 15985, CI 19140, CI 20040, CI 21100, CI 21108, CI 47000, CI 47005, green pigments with Color Index numbers CI 61565, CI 61570, CI 74260, orange pigments with Color Index numbers CI 11725, CI 15510, CI 45370, CI 71105, red pigments with Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

15. The process according to any one of claims 1 to 14, wherein agent (b) comprises at least one film-forming, hydrophobic polymer.

16. The process according to one of claims 1 to 15, characterized in that first the agent (a) is applied, then the agent (b) is applied, the period between the application of the agents (a) and (b) being at most 24 hours, preferably at most 12 hours, further preferably at most 6 hours and most preferably at most 30 minutes.

17. The process according to any of claims 1 to 16, comprising the following steps in the order indicated
(1) Application of agent (a) on the keratinous material,
(2) Allow the agent (a) to act for a period of 10 seconds to 10 minutes, preferably from 10 seconds to 5 minutes,
(3) if necessary, rinse the keratinous material with water,
(4) Application of agent (b) on the keratinous material,
(5) Allow the agent (b) to act for a period of 30 seconds to 30 minutes, preferably from 30 seconds to 10 minutes, and
(6) Rinse the keratinous material with water.

18. Kit-of-parts for dyeing keratinous material, comprising separately packaged
a first container with an agent (a), said agent (a) containing at least one organic silicon compound and at least one surfactant as described in claims 1 to 12, and
a second container with an agent (b), wherein the agent (b) contains at least one pigment as described in claims 1, 13, 14 and/or 15.
